(19) United States
(12) Patent Application Publication
(10) Pub. No.: US 2024/0041704 A1
(43) Pub. Date: Feb. 8, 2024

(54) NOVEL ANTICARIES MATERIAL FOR DENTAL USE

(71) Applicant: The University of Hong Kong, Hong Kong (CN)

(72) Inventors: Ollie YiRu YU, Hong Kong (CN); Ying Kei LUNG, Hong Kong (CN); Chun Hung CHU, Hong Kong (CN); Xing Yun GE, Hong Kong (CN)

(21) Appl. No.: 18/342,056

(22) Filed: Jun. 27, 2023

Related U.S. Application Data
(60) Provisional application No. 63/368,562, filed on Jul. 15, 2022.

(51) Int. Cl. A61K 6/889; A61K 6/853; A61K 6/887; A61K 6/844

(52) U.S. Cl. CPC A61K 6/889; A61K 6/853; A61K 6/887; A61K 6/844

(57) ABSTRACT

The subject invention pertains to a novel glass-ionomer cement (NGIC) that provides an alternative restorative material for dental amalgams being phased out; has improved mechanical properties compared to conventional glass-ionomer cement (GIC) products; has improved adhesive properties compared to conventional GIC products; provides sufficient biocompatibility; can release ions to promote remineralization of the teeth, which inhibits tooth decay; can inhibit growth of bacteria, which inhibits tooth decay; provides increased retention time and decreased frequency of replacement in the oral cavity. Formulations of the subject invention can include powders and solutions containing silicate glass, poly(vinylphosphonic acid) (PVPA), nanosilver bioactive glass, and polyacrylic acid.

### CROSS-REFERENCE TO RELATED APPLICATION

This application claims the benefit of U.S. Provisional Application Ser. No. 63/368,562, filed Jul. 15, 2022, which is hereby incorporated by reference in its entirety including any tables, figures, or drawings.

### BACKGROUND OF THE INVENTION

Conventional glass polyalkenoates or glass-ionomer cements (GIC), since their introduction to the dental arts, have occupied a very important position in dentistry, mainly as bonding, sealing and restorative materials. Conventional GIC was introduced by Wilson and Kent (1969) which is composed of powder and liquid calcium fluoroaluminate glass powder and an aqueous solution of the acrylic acid polymer [1].

Conventional GIC has several advantages, such as fluoride release for caries prevention or inhibition, ideal transparency during curing, similar thermal expansion coefficient as dentin [2]. However, it does not have antimicrobial effects. Its poor physical and mechanical properties, susceptibility to dehydration and moisture contamination also limit its clinical application and longevity [3].

### BRIEF SUMMARY OF THE INVENTION

Embodiments of the subject invention provide a novel dental material useful for the management of tooth decay (e.g., dental caries). Embodiments provide a restorative material useful for filling of tooth decay, a cement material for the cementation of other dental restorations to the tooth, or a sealant for the sealing of the grooves in tooth surface for caries prevention or inhibition, and in other applications. Embodiments provide a glass-ionomer-based dental material, i.e., a novel glass-ionomer cement (NGIC).

Embodiments of the subject invention provide an NGIC material that has antimicrobial properties and can inhibit the growth of microorganisms that can cause caries. In certain embodiments antimicrobial properties can be derived from or enhanced by silver nanoparticles or nanosilver bioactive glass. Embodiments of the NGIC material also release certain ions, which can harden the carious tooth structure. Embodiments of the provided dental material have good adhesive properties to tooth structure, allowing them to form a tight seal between the internal structures of the tooth and the material. These properties can reduce dental caries occurrence or inhibit dental caries. Embodiments have enhanced the anticaries properties, mechanical properties, and adhesive properties comparing to related art glass-ionomer cements.

Embodiments of the subject invention provide an NGIC material for multiple dental use (e.g., for caries management and filling or sealing of tooth decay). Embodiments can be used in a tooth with decay to restore the morphology and function of the tooth structure with cavitation, or to adhere other types of restoration to a tooth surface. Embodiments can also be used in healthy teeth for caries prevention, reduction, or inhibition. Embodiments providing the applied material in the oral cavity can act as a sustainable releasing system to release factors for caries prevention, reduction, or inhibition across some or all of the dentition.

Embodiments provide silver nanoparticles as broad-spectrum antimicrobial agents that can inhibit dental caries and enhance remineralization. Embodiments also provide bioactive glass (e.g., 45S5 bioactive glass) which has excellent biocompatibility and can be used as a clinical filling material due to its ability of remineralization to tooth hard tissue. The provided nanosilver bioactive glass can improve the caries inhibition effect, reduce mineral loss and promote remineralization of demineralized enamel and dentin, decrease or inhibit caries around a restoration, and decrease the frequency of restoration replacement.

Certain embodiments provide an improvement to a related art product such as conventional glass-ionomer cement (GIC). Conventional GIC has no antimicrobial properties and cannot prevent nor inhibit caries. Conventional GIC also has poor physical properties, has low fracture resistance, tends to facture with occlusal loading, has poor abrasion resistance, and tends to wear with daily tooth brushing. For at least these reasons, the longevity of conventional GIC is unsatisfactory after placement in the mouth.

Embodiments of the subject invention provide a novel antibacterial glass-ionomer cement (NGIC) material with antimicrobial properties, anti-caries function, improved mechanical properties, and improved adhesive properties to the tooth surface. These properties increase the retention time of the restoration in the mouth and decrease the frequency of restoration replacement.

Embodiments of NGIC provide a nanosilver bioactive glass and PVPA with biocompatibility, antimicrobial, and remineralizing properties. Embodiments with PVPA also enhance the mechanical properties and adhesive properties of the material. Certain embodiments greatly increase the antimicrobial properties, remineralizing properties, mechanical properties, and adhesive property of the provided NGIC material as compared to related art materials.

Embodiments advantageously provide an NGIC that provides an alternative restorative material for dental amalgams being phased out; has improved mechanical properties compared to conventional GIC products; has improved adhesive properties compared to conventional GIC products; provides sufficient biocompatibility; can release ions to promote remineralization of the teeth, which inhibits or prevents tooth decay; can inhibit growth of bacteria, which inhibits or prevents tooth decay; and provides increased retention time and decreased frequency of replacement in the oral cavity.

### BRIEF DESCRIPTION OF THE DRAWINGS

FIG. 1 is a chart of cell viability of human gingival fibroblast cells (HGF), showing effects of materials according to certain embodiments of the subject invention on the toxicity of HGFs. The CCk-8 results showed the viability of various group for 7 days. PVPA Groups have the same biocompatibility with the commercial GIC materials of Group B (e.g., Conventional GIC without PVPA, can also be referred to as 0% PVPA).

FIG. 2 contains two charts of compressive strength and diametral tensile strength of NGIC and GIC control, showing the results of the compressive strength and diametral tensile strength in various groups according to certain embodiments of the subject invention. Panel A shows compressive strength. Panel B shows diametral tensile strength. (*p<0.05, ***p<0.001).

FIG. 3 illustrates SEM images and EDX spectrum of a nanosilver bioactive glass (NanoAg BAG) according to an embodiment of the subject invention. (A) 2000× magnification view of a NanoAg BAG according to an embodiment of the subject invention showing granular particles; (B) 6000× magnification view of a NanoAg BAG according to an embodiment of the subject invention; (C) 10000× magnification view of a NanoAg BAG according to an embodiment of the subject invention; (D) EDX spectrum of a NanoAg BAG according to an embodiment of the subject invention. The SEM images in FIG. 3 showed the NanoAg BAG granular particles and EDX spectrum of the NanoAg BAG showed the peak of Ag around 3 KeV.

FIG. 4 is a chart of cell viability of human gingival fibroblast cells (HGF), showing effects of materials according to certain embodiments of the subject invention on the toxicity of HGFs. The CCk-8 results showed the viability of various group for 7 days (**p<0.01, ns p>0.05).

FIG. 5 contains two charts showing the results of the compressive strength and diametral tensile strength in various groups of materials according to certain embodiments of the subject invention. A. compressive strength. B. diametral tensile strength. (**p<0.01).

FIG. 6 is a chart showing fluoride release of NGIC materials containing NanoAg BAG and PVPA according to certain embodiments of the subject invention and a conventional GIC control group for 14 days.

FIG. 7 is a chart showing phosphate ion release of NGIC materials containing NanoAg BAG and PVPA according to certain embodiments of the subject invention and a conventional GIC control group for 14 days (**p<0.01).

FIG. 8 is a chart showing calcium ion release of NGIC materials containing NanoAg BAG and PVPA according to certain embodiments of the subject invention and control group for 14 days (**p<0.01).

FIG. 9 is a chart showing silver ion release of a conventional GIC and NGICs containing NanoAg BAG and PVPA according to certain embodiments of the subject invention for 14 days (**p<0.01).

FIG. 10 shows the results of the flexural strength in various groups of materials according to certain embodiments of the subject invention. (*p<0.01).

FIG. 11 is a chart of the biofilm metabolic activity, showing the antibacterial effect of NGIC in various groups of materials according to certain embodiments of the subject invention.

### DETAILED DISCLOSURE OF THE INVENTION

Compositions of NGIC in accordance with embodiments of the subject invention include powder and liquid components. The preparation of the NGIC samples is in certain embodiments conducted by a hand-mix of an NGIC powder and an NGIC liquid. The powder can contain nanosilver bioactive glass and/or the silicate glass-powder (e.g., calcium-alumino-fluorosilicate glass). The liquid can contain poly(vinylphosphonic acid) (PVPA) and/or polyacrylic acid. The liquid can be created from powdered components added to a liquid base prior to the powder and liquid mixing. The respective weight percentage of each component can be adjust based on the clinical application of the NGIC. Different formulations of powder and liquid elements can be used in different clinical situations.

TABLE 1
Exemplary components for an NGIC
NGIC

| NGIC-Powder | NGIC-Liquid |
|---|---|
| 90.7%-94.5% Fluoro- | 50% Deionized water |
| alumino-silicate glass powder | 40% Polyacrylic acid powder |
| 4.3%-4.5% polyacrylic acid powder | 10% PVPA solid |
| 1%-5% Nanosilver bioactive glass powder | |

Poly(vinylphosphonic acid) (PVPA) and its derivatives has the bifunctionality of the phosphonate group, which can form strong association with cations. PVPA is biocompatible. PVPA-modified surfaces enhanced adhesion, differentiation and mineralization of clonal osteogenic cells [4].

Embodiments advantageously employ PVPA to replace some portion or all related art polymer materials such as poly(acrylic acid), poly(acrylic-itaconic acid) and poly(acrylic-co-maleic acid) found in conventional GIC formulations [5]. In certain embodiments the replacement improves the mechanical properties as compared to related art conventional GIC compositions.

Silver nanoparticles, including nanosilver particles, are broad-spectrum antimicrobial agents that can be used to prevent, reduce, or inhibit dental caries [6]. Nanosilver particles means silver atoms clustered together to form particles 1-100 nm in size (e.g., 8-15 nm in particle size.) Due to the high surface area of the particles, they can attach to the outer cell membrane of bacteria, changing the permeability and cellular structure of the bacteria to kill bacterial cells [7].

Bioactive glass (e.g., 45S5 Bioglass®) has excellent biocompatibility [8]. 45S5 Bioglass® promotes the remineralization of tooth hard tissue, including enamel and dentin [9]. The glass particles also have antibacterial properties [10].

Embodiments of the subject invention provide numerous advantages over related art. For example, U.S. Pat. No. 5,179,135 (Ellis, et al.) teaches a poly(vinylphosphonic acid) glass-ionomer cement that is a simple mixture of conventional glass-ionomer cement material with poly(vinylphosphonic acid) solution and silicate glass powder. Embodiments of the subject invention advantageously use poly(vinylphosphonic acid) (PVPA) powder instead of liquid to improve mechanical strength of the material. Embodiments also add synthesized nanosilver bioactive glass, PVPA powder, and silicate glass powder to provide a novel composition with multiple favorable properties. The silver nanoparticles are broad-spectrum antimicrobial agents that can inhibit dental caries and enhance remineralization, while 45S5 bioactive glass has excellent biocompatibility, and provides a clinical filling material due to its ability to provide remineralization to dental tissue. Embodiments inhibit caries through more steady fluoride release over a prolonged period and reduce mineral loss and promote remineralization of demineralized enamel and dentin better than the conventional GIC materials. The provided nanosilver bioactive glass improves the caries inhibition effect, decrease the caries and mineral loss surrounding the restoration, promotes remineralization of demineralized enamel and dentin, and decreases the frequency of restoration replacement.

Embodiments provide a unique composition of dental material, which includes a mixture of fluoro-alumino-silicate glass powder along with nanosilver bioactive glass and a mixture of polyacrylic acid and PVPA solid. The provided composition is specifically designed to address the etiopathogenesis of dental caries, and provides multiple benefits such as improved mechanical strength, antibacterial and remineralizing properties. The use of GIC liquid mixed with PVPA solid instead of pure PVPA liquid is a novel approach that significantly enhances the mechanical strength of the material. Additionally, the incorporation of nanosilver bioactive glass and 45S5 bioactive glass enhances the antibacterial and remineralizing properties of the material, making it a highly effective dental material for precise caries management. In certain embodiments, the provided novel composition offers a significant improvement over related art and is an advancement in the field of dental caries management.

Embodiments advantageously incorporate synthesized nanosilver bioactive glass into dental materials. The provided silver nanoparticles are broad-spectrum antimicrobial agents that can be used to prevent dental caries and enhance remineralization. Additionally, 45S5 bioactive glass has excellent biocompatibility, and can be used as a clinical filling material due to its capacity for remineralization to form tooth hard tissue. The provided 45S5 bioactive glass can prevent caries through more steady fluoride release over a prolonged period and can reduce mineral loss and promote remineralization of demineralized enamel and dentin as compared to conventional GIC materials. The application of the nanosilver bioactive glass can improve the caries prevention effect, decrease the caries surrounding the restoration, decrease mineral loss, promote remineralization of demineralized enamel and dentin, and decrease the frequency of restoration replacement.

Embodiments provide a dental cement with antimicrobial properties to minimize the formation of new dental caries due to bacterial growth. The provided dental cement can also promote re-mineralization of the dental enamel. The antibacterial agent in certain embodiments is nanosilver which is a component of a bioactive glass. The bioactive glass can be 45S5 bioactive glass which is commercially available. The base dental cement can also be commercially available, meaning that the invention can be readily prepared from commercial materials that are already approved for human internal use. In one embodiment the composition is a mixture of a glass ionomer cement with a nanosilver bioactive glass. Such compositions exhibit good compressive and tensile strength.

Another example of how embodiments of the subject invention provide advantages over related art is seen with respect to United States Patent Application 2021/0212906 (Sakamoto, et al.) which teaches a Dental glass-ionomer cement composition comprised of non-crosslinked polyalkenoic acid and silicate glass powder. In contrast, embodiments of the subject invention provide nanosilver bioactive glass, silicate glass-powder, and polyacrylic acid added with poly(vinylphosphonic acid) (PVPA). The provided PVPA improves the mechanical strength for restoration, fatigue limit, and adhesive bond properties as well as prolonging the retention time of the restoration and decreasing the frequency of restoration replacement.

Another example of how embodiments of the subject invention provide advantages over related art is seen with respect to United States Patent Application 2007/0122356 (Kessler, et al.) which teaches an antimicrobial glass composition that includes silver in combination with a glass-ionomer cement. One difference between this related art and embodiments of the subject invention is the composition of the material. While the related art discloses a plastic-reinforced glass ionomer cement compomer that combines a composite with a glass ionomer and silver particles; embodiments of the subject invention provide a glass ionomer cement that contains nanosilver bioactive glass and PVPA. Comparing with compomers, the provided glass ionomer cement has a superior performance in fluoride releasing and thus a better remineralising effect. The use of nanosilver bioactive glass and PVPA in embodiments of the subject invention provides improved antibacterial properties and better overall performance compared to the related art.

Another example of how embodiments of the subject invention provide advantages over related art is seen with respect to U.S. Pat. No. 9,211,246 (Hack) which teaches bioactive glass with an average particle size of around 10 microns containing 0-15% of silver oxide.

The micron-size bioactive glass is not as effective with bacteria as the nano-size provided in certain embodiments of the subject invention, due at least in part to the reduced surface area. The related art's use of bioactive glass in a large size could damage the mechanical strength of the added materials, making it unsuitable for dental restoration. In addition, the related art materials used 0-15% of silver oxide. Silver oxide does not provide the same level of antibacterial properties as nanosilver bioactive glass. In addition, a high weight percentage of silver oxide can compromise material aesthetics, which is an important property for dental repair material.

The respective particle sizes of bioactive glass and silver are different. Embodiments of the subject invention provide bioactive glass of 50-120 nm in size incorporated with nanosilver of 8-15 nm in size. The incorporation of nanosilver into bioactive glass is beneficial and in certain embodiments necessary to achieve optimal antimicrobial properties while maintaining the mechanical strength and aesthetics of the material.

In certain embodiments, bioactive glass particles of 50-120 nm in size is important because biomaterials containing nanoparticles exhibit better bioactivity and higher mechanical stability compared to the micro-size bioactive glass particles. Bioactive glass particles larger than 120 nm in size have a slower bioactive response and fail to provide good mechanical properties in lower concentration.

In certain embodiments, nanosilver of 8-15 nm in size is important because the nano size particles have higher surface area than micro size particles, they can attach to the outer cell membrane of bacteria, changing the permeability and cellular structure of the bacteria to kill bacterial cells. Nanosilver smaller than 8 nm in size can be more cytotoxic due to the high surface area. Nanosilver larger than 15 nm in size are less efficient to inhibit bacteria.

Another example of how embodiments of the subject invention provide advantages over related art is seen with respect World Intellectual Property Organization Application WO 2022/058448 (deBarra, et al.) which teaches a non-nanosilver bioactive glass. Embodiments of the subject invention incorporate the nanosilver bioactive glass into the silicate glass powder, improving the mechanical and antibacterial properties of the NGIC compared to related art that teaches only how to improve the setting (e.g., working) time and flexural strength of the GIC.

Example 10 provides a non-limiting and exemplary embodiment of a therapeutic compound for dental use according to an embodiment of the subject invention.

In this embodiment, the composition (all percentages by weight) of nanosilver bioactive glass (NBG) is SiO₂ at 41.1%, CaO at 23.9%, Na₂O at 24.0%, P₂O₅ at 5.9%, and Ag nanoparticles at 5.1%. NBG is chemically stable in biological environment. It has antimicrobial property as well, since it elevates the pH and osmolarity locally, thereby creating unfavorable environment for bacterial growth. Other suitable or alternative values or ranges of compositions include values listed in the following paragraphs, taken alone or in various combinations.

In this composition the SiO₂ at 41.1% was selected to provide the bioactivity of the bioactive glass of NGIC. Higher amounts of SiO₂ can decrease the bioactivity. Lower amounts of SiO₂ can increase the bioactivity. Other suitable values or ranges include 43-47%.

In this composition the CaO at 23.9% was selected to provide remineralization effect of NGIC as it increased the calcium ion release. Higher amounts can increase the calcium release and remineralization effect. Lower amounts can have lower calcium release and decrease the remineralization effect. Other suitable values or ranges include 22.5-26.5%.

In this composition the Na₂O at 24.0% was selected to provide antibacterial properties of NGIC by increasing the local pH which is cytotoxic towards bacteria and promotes or accelerates glass degradation. Higher amounts can increase the antibacterial effect and glass degradation but can also increase the cell cytotoxicity. Lower amounts can decrease the antibacterial effect and glass degradation. Other suitable values or ranges include 22.5-26.5%.

In this composition the P₂O₅ at 5.9% was selected to provide mineralization according to Ca:P ratio, as it increased the phosphate ion release to promote the apatite formation and induce the crystallization of calcium phosphate phases. Higher amounts can inhibit the bioactivity. Lower amounts can lower the rate of apatite formation. Other suitable values or ranges include 5-7%.

In this composition the Ag nanoparticles at 5.1% were selected to provide antibacterial effect and biocompatibility of NGIC. Higher amounts can provide better antibacterial effect but can also increase the cell cytotoxicity. Lower amounts can provide decrease antibacterial effect and cell cytotoxicity. Other suitable values or ranges include 5-10%.

In this composition the Ag nanoparticles size range of 8-15 nm was selected to provide better antibacterial effect of NGIC. Higher (Larger) size of Ag can increase the antibacterial effect but it can also increase the cell cytotoxic effect. Lower (Smaller) size of Ag can increase the antibacterial effect decrease the antibacterial effect but it can decrease the cell cytotoxic effect.

In this composition the NBG particles were formed in a size range of 50-120 nm to provide suitable mechanical strength and antibacterial effect. Higher size of NBG particles can decreased the mechanical strength. Lower size of NBG particles are less efficient to inhibit bacteria.

In this composition the NBG was mixed with an NGIC as described below in a range of ratios between ((1% NBG) to (99% NGIC)) and ((5% NBG) to (95% NGIC)) to provide suitable mechanical strength and antibacterial effect of a biocompatibility NGIC. Higher amounts of NBG can provide better antibacterial effect but lower mechanical strength and increased cell cytotoxicity. Lower amounts of NBG can provide lower antibacterial effect and mechanical strength and decreased cell cytotoxicity. In particular a ratio of 1% NBG to 99% NGIC was found to provide highest mechanical strength (e.g., compressive strength and diametral tensile strength were significantly improved) and similar biocompatibility with commercial GIC. A ratio of 2% NBG to 98% NGIC was found to provide lower mechanical strength, increased cell cytotoxicity than 1% NBG to 99% NGIC (but still higher than the strength of 5% NBG to 95% NGIC) and more Ag, Ca, P release. A ratio of 5% NBG to 95% NGIC was found to provide lower mechanical strength, increased cell cytotoxicity than either (1% NBG to 99% NGIC) or (2% NBG to 98% NGIC) and more Ag, Ca, P release.

In this embodiment the NGIC Powder included SiO₂ at 0.41-2.055 wt % (e.g., corresponds to bioactive glass at 1-5 wt %), CaO at 0.239-1.195 wt %, Na₂O at 0.24-1.2 Wt %, P₂O₅ at 0.59-0.295 wt %, Ag nanoparticles at 0.51-0.255 wt %, SiO₂ at 33.4-41.48 wt % (e.g., corresponds to GIC powder at 99-95 wt %), Al₂O₃ at 9.09-28.314 wt %, AlF₃ at 1.52-2.376 wt %, CaF₂, 14.915-19.899 wt %, NaF at 3.42-9.207 wt %, and AlPO₄ at 3.61-11.88 wt %.

In this composition the SiO₂ at 0.41-2.055 wt % (e.g., the SiO₂ composition in Ag bioactive glass, i.e., Ag45S5 bioactive glass) was selected to provide suitable mechanical strength and antibacterial effect of a biocompatible NGIC. Higher amounts can provide better antibacterial effect but lower mechanical strength and increased cell cytotoxicity. Lower amounts can provide lower antibacterial effect and mechanical strength and decreased cell cytotoxicity.

In this composition the CaO at 0.239-1.195 wt % was selected to provide suitable mechanical strength and antibacterial effect of a biocompatible NGIC. Higher amounts can provide better antibacterial effect but lower mechanical strength and increased cell cytotoxicity. Lower amounts can provide lower antibacterial effect and mechanical strength and decreased cell cytotoxicity.

In this composition the Na₂O at 0.24-1.2 wt % was selected to provide suitable mechanical strength and antibacterial effect of a biocompatible NGIC. Higher amounts can provide better antibacterial effect but lower mechanical strength and increased cell cytotoxicity. Lower amounts can provide lower antibacterial effect and mechanical strength and decreased cell cytotoxicity.

In this composition the P₂O₅ at 0.59-0.295 wt % was selected to provide suitable mechanical strength and antibacterial effect of a biocompatible NGIC. Higher amounts can provide better antibacterial effect but lower mechanical strength and increased cell cytotoxicity. Lower amounts can provide lower antibacterial effect and mechanical strength and decreased cell cytotoxicity.

In this composition the P₂O₅ at 0.59-0.295 wt % was selected to provide suitable mechanical strength and antibacterial effect of a biocompatible NGIC. Higher amounts can provide better antibacterial effect but lower mechanical strength and increased cell cytotoxicity. Lower amounts can provide lower antibacterial effect and mechanical strength and decreased cell cytotoxicity.

In this composition the Ag nanoparticles at 0.51-0.255 wt % (e.g., added to the NGIC as part of the Ag substituted NBG) was selected to provide suitable mechanical strength and antibacterial effect of a biocompatible NGIC. Higher amounts can provide better antibacterial effect but lower mechanical strength and increased cell cytotoxicity. Lower amounts can provide lower antibacterial effect and mechanical strength and decreased cell cytotoxicity.

In this composition the SiO₂ at 33.4-41.48 wt % was selected to provide SiO₄⁴⁻ release after the acid-base reaction of NGIC. Higher amounts can increase the SiO₄⁴⁻ release. Lower amounts can decrease the SiO₄⁴⁻ release.

In this composition the Al₂O₃ at 9.09-28.314 wt % was selected to provide setting of cement formation by forming negative sites in silica glass network. Higher amounts can increase the aluminum ion release. Lower amounts can decrease the aluminum ion release.

In this composition the AlF₃ at 1.52-2.376 wt % was selected to provide fluoride ion release after the acid-base reaction of NGIC. Higher amounts can increase the fluoride ion release. Lower amounts can decrease the fluoride ion release.

In this composition the CaF₂, 14.915-19.899 wt % was selected to provide calcium and fluoride ion release during the acid-base reaction of NGIC. Higher amounts can increase calcium and fluoride ion release. Lower amounts can decrease the calcium and fluoride ion release.

In this composition the NaF at 3.42-9.207 wt % was selected to provide sodium and fluoride ion release after the acid-base reaction of NGIC. Higher amounts can increase the sodium and fluoride ion release. Lower amounts can decrease the sodium and fluoride release.

In this composition the AlPO₄ at 3.61-11.88 wt % was selected to provide phosphate ion release after the acid-base reaction of NGIC. Higher amounts can increase the phosphate ion release. Lower amounts can decrease the phosphate ion release.

In this embodiment the NGIC Liquid included deionized water at 45 wt %, Polyacrylic acid at 36 wt %, Polybasic carboxylic acid at 9 wt %, and Poly (vinylphosphonic acid) at 10 wt %.

In this composition the deionized water at 45 wt % was selected to provide as a solvent, for the dissolution of the polyacrylic acid, and allows it to ionize and donate protons, thereby behaving as a Bronsted-Lowry acid. Higher amounts can speed up the acid-base reaction of NGIC. Lower amounts can delay the acid-base reaction of NGIC.

In this composition the Polyacrylic acid at 36 wt % was selected to provide higher mechanical strength and suitable setting time. Higher amounts can decrease the mechanical strength of NGIC. Lower amounts can make the NGIC difficult to mix and lead to lower mechanical strength.

In this composition the Polybasic carboxylic acid at 9 wt % was selected to provide improved handling properties by extending the working time and reducing the setting time. Higher amounts can increase the working time and reducing the setting time. Lower amounts can decrease the working time and increase the setting time.

In this composition the Poly (vinylphosphonic acid) at 10 wt % was selected to optimum mechanical properties of the NGIC. Higher amounts cause difficult mixing of the GIC liquid and PVPA solid. Lower amounts would not improve the mechanical strength.

Specific structural, measurable, or quantifiable differences between a GIC solution as known in related art and the NGIC Solid plus NGIC Liquid formulations provided by embodiments of the subject invention include the following. For PVPA, solid state 31P NMR can detect the PVPA functional group, (OH)2P═O (acid group). The chemical shift is about 33 ppm. For Ag, XPS can detect and quantify the amount of Ag in a product.

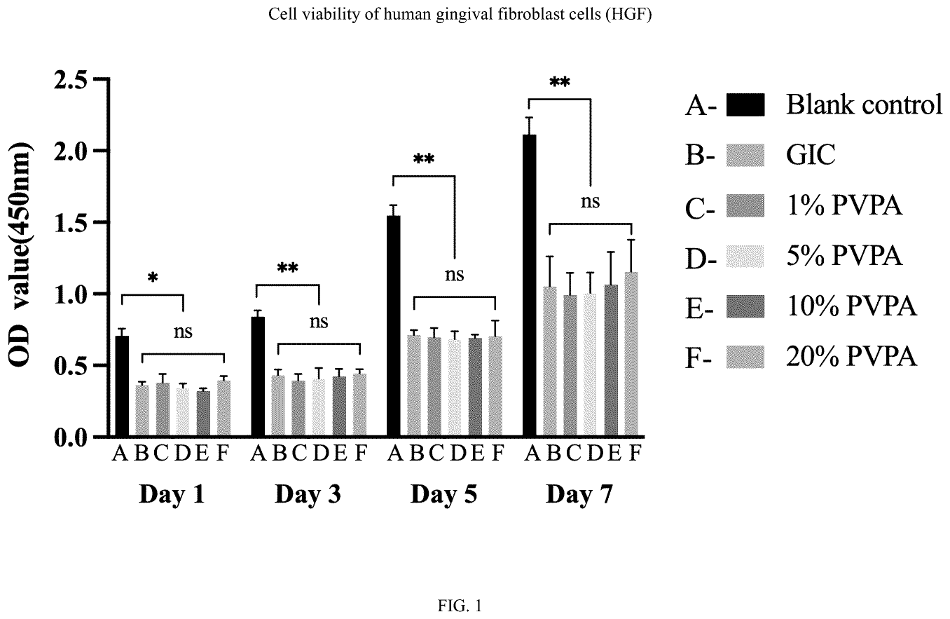

Turning now to the figures, FIG. 1 is a chart showing effects of materials according to certain embodiments of the subject invention on the optical density (OD) of the cell culture medium after cell counting kit-8 (CCK-8) treatment at 450 nm, which represents the number of viable human gingival fibroblast cells (HGF) in the culture medium. The cell counting kit-8 (CCK-8) results showed the viability of blank control, 0% PVPA, 1% PVPA, 5% PVPA, 10% PVPA, and 20% PVPA groups at 1, 3, 5, and 7 days. PVPA Groups have the same biocompatibility with the commercial GIC materials (e.g., represented by 0% PVPA).

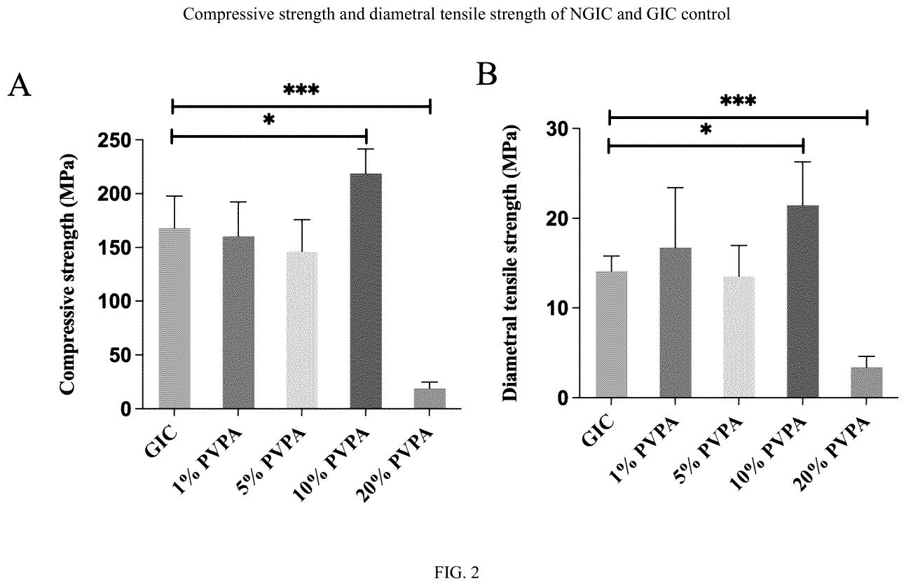

FIG. 2 contains two charts showing the results of the compressive strength and diametral tensile strength in 0% PVPA, 1% PVPA, 5% PVPA, 10% PVPA, and 20% PVPA groups according to certain embodiments of the subject invention. Panel A shows compressive strength and panel B shows diametral tensile strength. (*p<0.05, ***p<0.001).

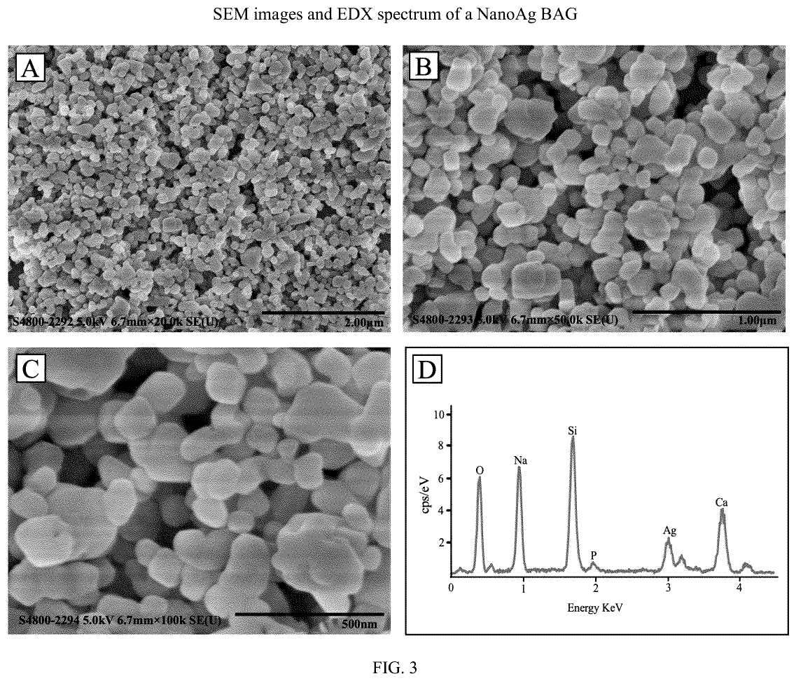

FIG. 3 illustrates scanning electron microscopy (SEM) images and EDX spectrum of a NanoAg BAG according to an embodiment of the subject invention. (A) 2000× magnification view of a NanoAg BAG according to an embodiment of the subject invention showing granular particles; (B) 6000× magnification view of a NanoAg BAG according to an embodiment of the subject invention; (C) 10000× magnification view of a NanoAg BAG according to an embodiment of the subject invention; (D) EDX spectrum of a NanoAg BAG according to an embodiment of the subject invention. SEM images showed the NanoAg BAG granular particles and EDX spectrum of the NanoAg BAG showed the peak of Ag.

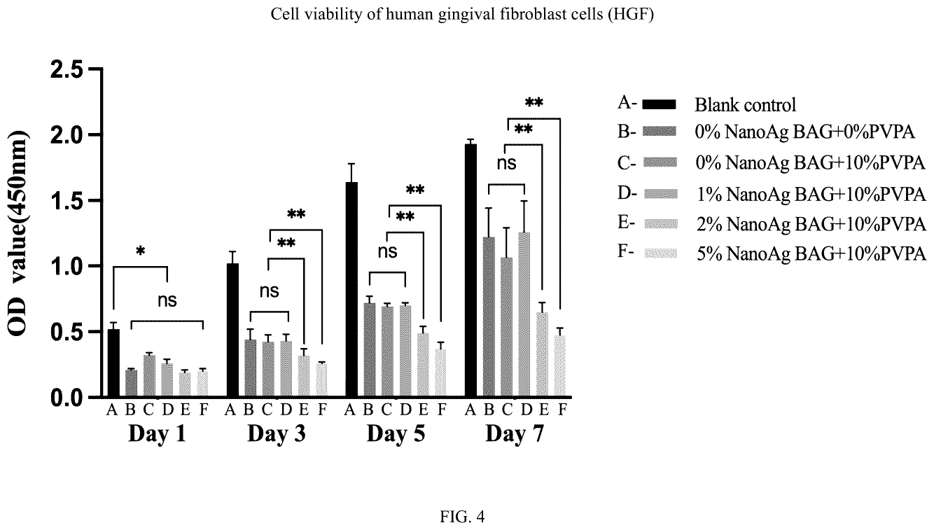

FIG. 4 is a chart showing effects of materials according to certain embodiments of the subject invention on the optical density (OD) of the cell culture medium after cell counting kit-8 (CCK-8) treatment at 450 nm, which represents the number of viable human gingival fibroblast cells (HGF) in the culture medium, for blank control, 0% NanoAg BAG+0% PVPA, 0% NanoAg BAG+10% PVPA, 1% NanoAg BAG+10% PVPA, 2% NanoAg BAG+10% PVPA, and 5% NanoAg BAG+10% PVPA groups at 1, 3, 5, and 7 days. The CCk-8 results showed the viability of various group for 7 days (**p<0.01, ns p>0.05).

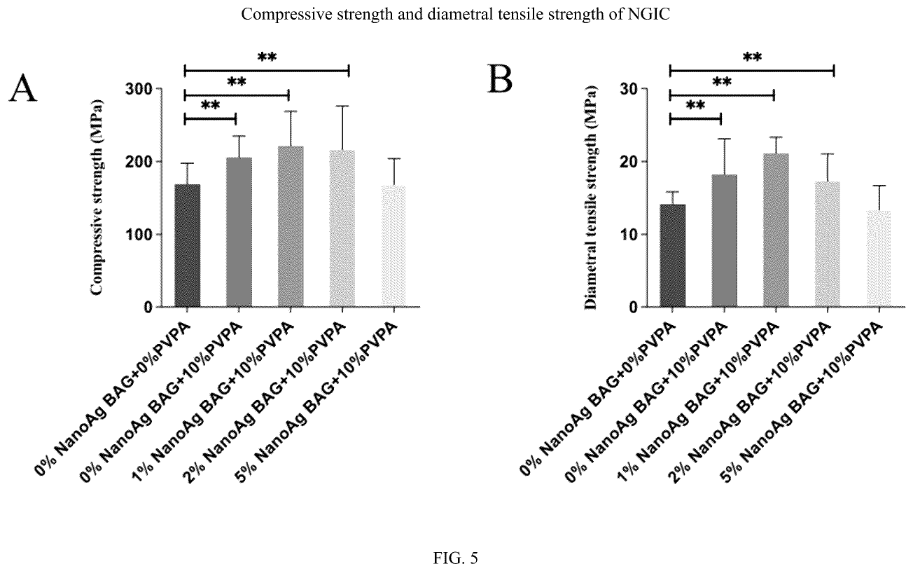

FIG. 5 contains two charts showing the results of the compressive strength and diametral tensile strength in 0% NanoAg BAG+0% PVPA, 0% NanoAg BAG+10% PVPA, 1% NanoAg BAG+10% PVPA, 2% NanoAg BAG+10% PVPA, and 5% NanoAg BAG+10% PVPA groups of materials according to certain embodiments of the subject invention. Panel A shows compressive strength and panel B shows diametral tensile strength. (**p<0.01).

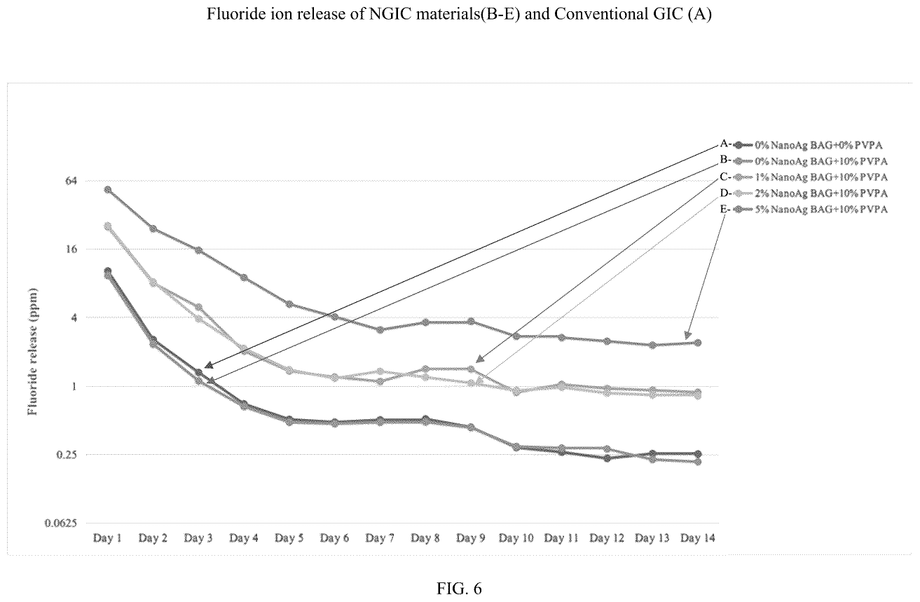

FIG. 6 is a chart showing fluoride release of NGIC materials containing NanoAg BAG and PVPA in 0% NanoAg BAG+10% PVPA, 1% NanoAg BAG+10% PVPA, 2% NanoAg BAG+10% PVPA, and 5% NanoAg BAG+10% PVPA groups according to certain embodiments of the subject invention and a control group (0% NanoAg BAG+0% PVPA) for 14 days.

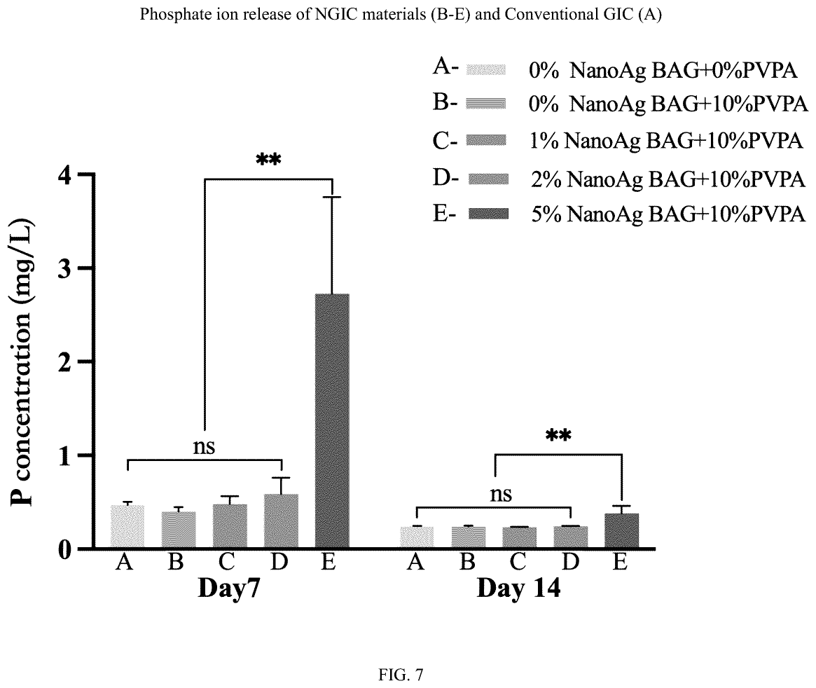

FIG. 7 is a chart showing phosphate ion release of NGIC materials containing NanoAg BAG and PVPA in 0% NanoAg BAG+10% PVPA, 1% NanoAg BAG+10% PVPA, 2% NanoAg BAG+10% PVPA, and 5% NanoAg BAG+10% PVPA groups according to certain embodiments of the subject invention and a control group (0% NanoAg BAG+0% PVPA) at 7 days and 14 days (**p<0.01).

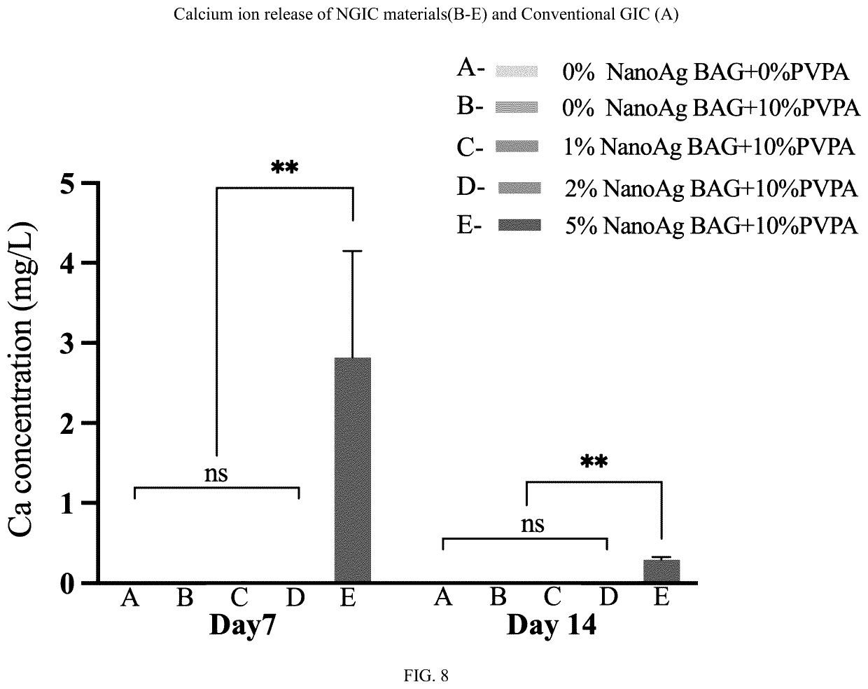

FIG. 8 is a chart showing calcium ion release of NGIC materials containing NanoAg BAG and PVPA in 0% NanoAg BAG+10% PVPA, 1% NanoAg BAG+10% PVPA, 2% NanoAg BAG+10% PVPA, and 5% NanoAg BAG+10% PVPA groups according to certain embodiments of the subject invention and a control group (0% NanoAg BAG+0% PVPA) at 7 days and 14 days (**p<0.01).

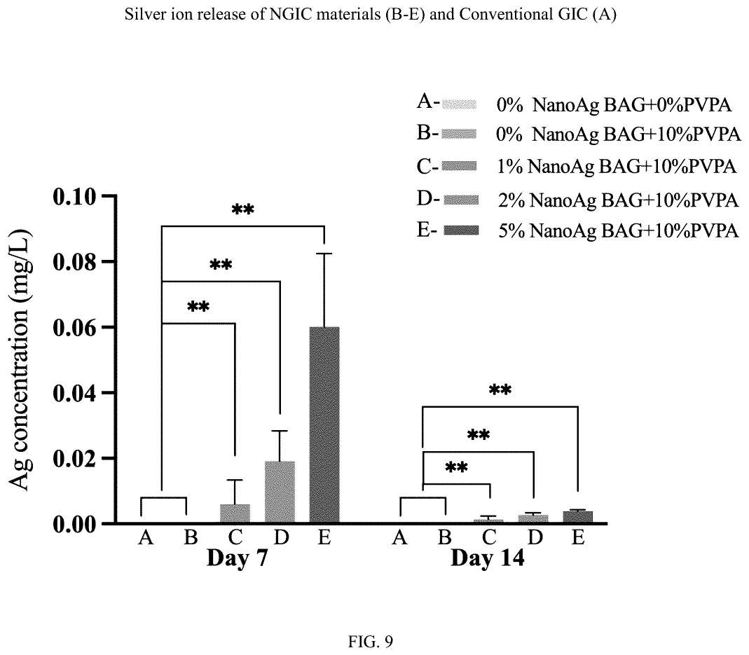

FIG. 9 is a chart showing silver ion release of a conventional GIC (0% NanoAg BAG+0% PVPA) and NGICs containing NanoAg BAG and PVPA in 0% NanoAg BAG+10% PVPA, 1% NanoAg BAG+10% PVPA, 2% NanoAg BAG+10% PVPA, and 5% NanoAg BAG+10% PVPA groups according to certain embodiments of the subject invention at 7 days and 14 days (**p<0.01).

### EXEMPLIFIED EMBODIMENTS

The invention may be better understood by reference to certain illustrative examples, including but not limited to the following:

Embodiment 1. A novel glass-ionomer cement (NGIC), comprising:

a silicate glass powder;

a polyacrylic acid powder;

a poly(vinylphosphonic acid) (PVPA) powder;

a nanosilver bioactive glass; and

a polyacrylic acid solution.

Embodiment 2. The NGIC according to Embodiment 1, wherein the silicate glass-powder comprises calcium-alumino-fluorosilicate glass.

Embodiment 3. The NGIC according to Embodiment 2, wherein the silicate glass-powder is calcium-alumino-fluorosilicate glass.

Embodiment 4. The NGIC according to Embodiment 1, wherein the wt % ratio of (polyacrylic acid)/PVPA is greater than 99/1.

Embodiment 5. The NGIC according to Embodiment 1, wherein the wt % ratio of (polyacrylic acid)/PVPA is greater than 95/5, but less than or equal to 99/1.

Embodiment 6. The NGIC according to Embodiment 1, wherein the wt % ratio of (polyacrylic acid)/PVPA is greater than 90/10, but less than or equal to 95/5.

Embodiment 7. The NGIC according to Embodiment 1, wherein the wt % ratio of (polyacrylic acid)/PVPA is greater than 80/20, but less than or equal to 90/10.

Embodiment 8. The NGIC according to Embodiment 1, wherein the wt % ratio of (silicate glass powder)/(nanosilver bioactive glass) is greater than 99/1.

Embodiment 9. The NGIC according to Embodiment 1, wherein the wt % ratio of (silicate glass powder)/(nanosilver bioactive glass) is greater than 98/2, but less than or equal to 99/1.

Embodiment 10. The NGIC according to Embodiment 1, wherein the wt % ratio of (silicate glass powder)/(nanosilver bioactive glass) is greater than 95/5, but less than or equal to 98/2.

Embodiment 11. The NGIC according to Embodiment 1, wherein the wt % ratio of (polyacrylic acid)/PVPA is about 90/10, and the wt % ratio of (silicate glass powder)/(nanosilver bioactive glass) is greater than 95/5, but less than or equal to 99/1.

Embodiment 12. A therapeutic agent, comprising:

a novel glass-ionomer cement (NGIC), comprising a blended mixture of:

a dry powder comprising a silicate glass powder, a polyacrylic acid powder, and nanosilver bioactive glass; and

a solution containing polyacrylic acid and poly(vinylphosphonic acid) (PVPA).

Embodiment 13. The therapeutic agent according to Embodiment 12, wherein the silicate glass powder comprises calcium-alumino-fluorosilicate glass.

Embodiment 14. The therapeutic agent according to Embodiment 12, wherein the silicate glass powder is calcium-alumino-fluorosilicate glass.

Embodiment 15. The therapeutic agent according to Embodiment 12, wherein the wt % ratio of (polyacrylic acid)/PVPA is greater than 99/1.

Embodiment 16. The therapeutic agent according to Embodiment 12, wherein the wt % ratio of (polyacrylic acid)/PVPA is greater than 95/5, but less than or equal to 99/1.

Embodiment 17. The therapeutic agent according to Embodiment 12, wherein the wt % ratio of (polyacrylic acid)/PVPA is greater than 90/10, but less than or equal to 95/5.

Embodiment 18. The therapeutic agent according to Embodiment 12, wherein the wt % ratio of (polyacrylic acid)/PVPA is greater than 80/20, but less than or equal to 90/10.

Embodiment 19. The therapeutic agent according to Embodiment 12, wherein the wt % ratio of (silicate glass powder)/(nanosilver bioactive glass) is greater than 99/1.

Embodiment 20. The therapeutic agent according to Embodiment 12, wherein the wt % ratio of (silicate glass powder)/(nanosilver bioactive glass) is greater than 98/2, but less than or equal to 99/1.

Embodiment 21. The therapeutic agent according to Embodiment 12, wherein the wt % ratio of (silicate glass powder)/(nanosilver bioactive glass) is greater than 95/5, but less than or equal to 98/2.

Embodiment 22. The therapeutic agent according to Embodiment 12, wherein the wt % ratio of (polyacrylic acid)/PVPA is about 90/10, and the wt % ratio of (silicate glass powder)/(nanosilver bioactive glass) is greater than 95/5, but less than or equal to 99/1.

Embodiment 23. A method of preparing a NGIC, comprising combining a liquid mixture of polyacrylic acid and PVPA with a powdered mixture of silicate glass powder, polyacrylic acid powder and nanosilver bioactive glass.

Embodiment 24. The method according to Embodiment 23, wherein the silicate glass powder comprises calcium-alumino-fluorosilicate glass.

Embodiment 25. The method according to Embodiment 23, wherein the silicate glass powder is calcium-alumino-fluorosilicate glass.

Embodiment 26. The method according to Embodiment 23, wherein the wt % ratio of (polyacrylic acid)/PVPA is greater than 99/1.

Embodiment 27. The method according to Embodiment 23, wherein the wt % ratio of (polyacrylic acid)/PVPA is greater than 95/5, but less than or equal to 99/1.

Embodiment 28. The method according to Embodiment 23, wherein the wt % ratio of (polyacrylic acid)/PVPA is greater than 90/10, but less than or equal to 95/5.

Embodiment 29. The method according to Embodiment 23, wherein the wt % ratio of (polyacrylic acid)/PVPA is greater than 80/20, but less than or equal to 90/10.

Embodiment 30. The method according to Embodiment 23, wherein the wt % ratio of (silicate glass powder)/(nanosilver bioactive glass) is greater than 99/1.

Embodiment 31. The method according to Embodiment 23, wherein the wt % ratio of (silicate glass powder)/(nanosilver bioactive glass) is greater than 98/2, but less than or equal to 99/1.

Embodiment 32. The method according to Embodiment 23, wherein the wt % ratio of (silicate glass powder)/(nanosilver bioactive glass) is greater than 95/5, but less than or equal to 98/2.

Embodiment 33. The method according to Embodiment 23, wherein the wt % ratio of (polyacrylic acid)/PVPA is about 90/10, and the wt % ratio of (silicate glass powder)/(nanosilver bioactive glass) is greater than 95/5, but less than or equal to 99/1.

Embodiment 34. A method of treating dental caries in a subject, comprising:

providing a therapeutic agent comprising:

a novel glass-ionomer cement (NGIC), comprising:

silicate glass powder;

nanosilver bioactive glass;

polyacrylic acid powder; and

poly(vinylphosphonic acid) (PVPA); and

applying the therapeutic agent to at least one surface within an oral cavity of the subject.

Embodiment 35. The method according to Embodiment 34, wherein the silicate glass powder comprises calcium-alumino-fluorosilicate glass.

Embodiment 36. The method according to Embodiment 34, wherein the silicate glass powder is calcium-alumino-fluorosilicate glass.

Embodiment 37. The method according to Embodiment 34, wherein the wt % ratio of (polyacrylic acid)/PVPA is greater than 99/1.

Embodiment 38. The method according to Embodiment 34, wherein the wt % ratio of (polyacrylic acid)/PVPA is greater than 95/5, but less than or equal to 99/1.

Embodiment 39. The method according to Embodiment 34, wherein the wt % ratio of (polyacrylic acid)/PVPA is greater than 90/10, but less than or equal to 95/5.

Embodiment 40. The method according to Embodiment 34, wherein the wt % ratio of (polyacrylic acid)/PVPA is greater than 80/20, but less than or equal to 90/10.

Embodiment 41. The method according to Embodiment 34, wherein the wt % ratio of (silicate glass powder)/(nanosilver bioactive glass) is greater than 99/1.

Embodiment 42. The method according to Embodiment 34, wherein the wt % ratio of (silicate glass powder)/(nanosilver bioactive glass) is greater than 98/2, but less than or equal to 99/1.

Embodiment 43. The method according to Embodiment 34, wherein the wt % ratio of (silicate glass powder)/(nanosilver bioactive glass) is greater than 95/5, but less than or equal to 98/2.

Embodiment 44. The method according to Embodiment 34, wherein the wt % ratio of (polyacrylic acid)/PVPA is about 90/10, and the wt % ratio of (silicate glass powder)/(nanosilver bioactive glass) is greater than 95/5, but less than or equal to 99/1.

Embodiment 45. The method according to Embodiment 34, wherein the therapeutic agent further comprises a second solvent, a thickener, a buffer, and/or an oil.

Embodiment 46. The method according to Embodiment 34, wherein applying comprises rinsing, spraying dropping, brushing, injecting, or any combination thereof.

Embodiment 47. The method according to Embodiment 34, further comprising cross-linking wherein the therapeutic agent further comprises an oil comprising at least one component with a cross-linking functionality and optionally comprising a cross-linking agent.

Embodiment 48. A nanosilver bioactive glass, comprising:
about 41% SiO₂,
about 24% CaO,
about 24% Na₂O,
about 6% P₂O₅, and
about 5% Ag nanoparticles.

Embodiment 49. A nanosilver bioactive glass, comprising:
41% SiO₂,
24% CaO,
24% Na₂O,
6% P₂O₅, and
5% Ag nanoparticles.

Embodiment 50. A nanosilver bioactive glass, comprising:
about 41.1% SiO₂,
about 23.9% CaO,
about 24.0% Na₂O,
about 5.9% P₂O₅, and
about 5.1% Ag nanoparticles.

Embodiment 51. A nanosilver bioactive glass, comprising:
41.1% SiO₂,
23.9% CaO,
24.0% Na₂O,
5.9% P₂O₅, and
5.1% Ag nanoparticles.

Embodiment 52. A therapeutic agent, comprising:

a novel glass-ionomer cement (NGIC), comprising a blended mixture of:
a dry powder comprising Ag nanoparticles in an amount from 0.51-0.255 wt % and at least two of the following:
   SiO₂ in an amount from 0.41-2.055 wt %,
   CaO in an amount from 0.239-1.195 wt %,
   Na₂O in an amount from 0.24-1.2 wt %,
   P₂O₅ in an amount from 0.59-0.295 wt %,
   SiO₂ in an amount from 33.4-41.48 wt %,
   Al₂O₃ in an amount from 19.09-28.314 wt %,
   AlF₃ in an amount from 1.52-2.376 wt %,
   CaF₂ in an amount from 14.915-19.899 wt %,
   NaF in an amount from 3.42-9.207 wt %, and
   AlPO₄ in an amount from 3.61-11.88 wt %; and

a solution containing about 36 wt % polyacrylic acid and about 10 wt % poly(vinylphosphonic acid) (PVPA).

Embodiment 53. A therapeutic agent, comprising:

a novel glass-ionomer cement (NGIC), comprising a blended mixture of:
a dry powder comprising:
   Ag nanoparticles in an amount from 0.51-0.255 wt %,
   SiO₂ in an amount from 0-2.055 wt %,
   CaO in an amount from 0-1.195 wt %,
   Na₂O in an amount from 0-1.2 wt %,
   P₂O₅ in an amount from 0-0.295 wt %,
   SiO₂ in an amount from 33.4-41.48 wt %,
   Al₂O₃ in an amount from 0-28.314 wt %,
   AlF₃ in an amount from 0-2.376 wt %,
   CaF₂ in an amount from 14.915-19.899 wt %,
   NaF in an amount from 3.42-9.207 wt %, and
   AlPO₄ in an amount from 0-11.88 wt %; and
a solution comprising:
   45 wt % H₂O,
   36 wt % polyacrylic acid,
   9 wt % polybasic carboxylic acid, and
   10 wt % poly(vinylphosphonic acid) (PVPA).

Embodiment 54. A therapeutic agent, comprising:

a novel glass-ionomer cement (NGIC), comprising a blended mixture of:
a dry powder comprising:
   Ag nanoparticles in an amount from 0.51-0.255 wt %,
   SiO2 in an amount from 0.41-2.055 wt %,
   CaO in an amount from 0.239-1.195 wt %,
   Na₂O in an amount from 0.24-1.2 wt %,
   P₂O₅ in an amount from 0.59-0.295 wt %,
   SiO₂ in an amount from 33.4-41.48 wt %,
   Al₂O₃ in an amount from 19.09-28.314 wt %,
   AlF₃ in an amount from 1.52-2.376 wt %,
   CaF₂ in an amount from 14.915-19.899 wt %,
   NaF in an amount from 3.42-9.207 wt %, and
   AlPO₄ in an amount from 3.61-11.88 wt %; and
a solution comprising:
   45 wt % H₂O,
   36 wt % polyacrylic acid,
   9 wt % polybasic carboxylic acid, and
   10 wt % poly(vinylphosphonic acid) (PVPA).

Embodiment 55. A therapeutic agent comprising a mixture of:
a polyacrylic acid liquid;
a poly(vinylphosphonic acid) (PVPA) solid;
a nanosilver bioactive glass (NBG); and
a silicate glass powder.

Embodiment 56. The therapeutic agent according to Embodiment 55, the nanosilver bioactive glass comprising nanosilver particles of 8-15 nm in size.

Embodiment 57. The therapeutic agent according to Embodiment 55, the nanosilver bioactive glass comprising a population of nanosilver particles, a majority of which are between 8-15 nm in size.

Embodiment 58. The therapeutic agent according to Embodiment 55, the nanosilver bioactive glass comprising a population of nanosilver particles, 90% of which are between 8-15 nm in size.

Embodiment 59. The therapeutic agent according to Embodiment 55, the nanosilver bioactive glass comprising a population of nanosilver particles, 99% of which are between 8-15 nm in size.

Embodiment 60. The therapeutic agent according to Embodiment 55, the nanosilver bioactive glass comprising a population of nanosilver particles consisting essentially of nanosilver particles of 8-15 nm in size.

Embodiment 61. The therapeutic agent according to Embodiment 55, the nanosilver bioactive glass comprising a population of nanosilver particles consisting of nanosilver particles of 8-15 nm in size.

Embodiment 62. The therapeutic agent according to Embodiment 56, the bioactive glass comprising bioactive glass particles of 5-120 nm in size.

Embodiment 63. The therapeutic agent according to Embodiment 56, the bioactive glass comprising a population of bioactive glass particles, a majority of which are between 5-120 nm in size.

Embodiment 64. The therapeutic agent according to Embodiment 56, the bioactive glass comprising a population of 45S5 bioactive glass particles, 90% of which are between 5-120 nm in size.

Embodiment 65. The therapeutic agent according to Embodiment 56, the bioactive glass comprising a population of 45S5 bioactive glass particles, 99% of which are between 5-120 nm in size.

Embodiment 66. The therapeutic agent according to Embodiment 56, the bioactive glass comprising a population of 45S5 bioactive glass particles consisting essentially of 45S5 bioactive glass particles between 5-120 nm in size.

Embodiment 67. The therapeutic agent according to Embodiment 56, the bioactive glass comprising a population of 45S5 bioactive glass particles consisting of 45S5 bioactive glass particles between 5-120 nm in size.

Embodiment 68. The therapeutic agent according to Embodiment 57, the bioactive glass comprising bioactive glass particles of 5-120 nm in size.

Embodiment 69. The therapeutic agent according to Embodiment 57, the bioactive glass comprising a population of bioactive glass particles, a majority of which are between 5-120 nm in size.

Embodiment 70. The therapeutic agent according to Embodiment 57, the bioactive glass comprising a population of 45S5 bioactive glass particles, 90% of which are between 5-120 nm in size.

Embodiment 71. The therapeutic agent according to Embodiment 57, the bioactive glass comprising a population of 45S5 bioactive glass particles, 99% of which are between 5-120 nm in size.

Embodiment 72. The therapeutic agent according to Embodiment 57, the bioactive glass comprising a population of 45S5 bioactive glass particles consisting essentially of 45S5 bioactive glass particles between 5-120 nm in size.

Embodiment 73. The therapeutic agent according to Embodiment 57, the bioactive glass comprising a population of 45S5 bioactive glass particles consisting of 45S5 bioactive glass particles between 5-120 nm in size.

Embodiment 74. The therapeutic agent according to Embodiment 60, the bioactive glass comprising bioactive glass particles of 5-120 nm in size.

Embodiment 75. The therapeutic agent according to Embodiment 60, the bioactive glass comprising a population of bioactive glass particles, a majority of which are between 5-120 nm in size.

Embodiment 76. The therapeutic agent according to Embodiment 60, the bioactive glass comprising a population of 45S5 bioactive glass particles, 90% of which are between 5-120 nm in size.

Embodiment 77. The therapeutic agent according to Embodiment 60, the bioactive glass comprising a population of 45S5 bioactive glass particles, 99% of which are between 5-120 nm in size.

Embodiment 78. The therapeutic agent according to Embodiment 60, the bioactive glass comprising a population of 45S5 bioactive glass particles consisting essentially of 45S5 bioactive glass particles between 5-120 nm in size.

Embodiment 79. The therapeutic agent according to Embodiment 61, the bioactive glass comprising a population of 45S5 bioactive glass particles consisting of 45S5 bioactive glass particles between 5-120 nm in size.

Embodiment 80. The therapeutic agent according to Embodiment 61, the ratio of (NBG:GIC liquid) between (1 wt %:99 wt %) and (5 wt %:95 wt %), inclusive.

Embodiment 81. The therapeutic agent according to Embodiment 61, the (GIC) liquid comprising water and an NGIC powder.

Embodiment 82. The therapeutic agent according to Embodiment 61, the (GIC) liquid consisting essentially of water and an NGIC powder.

Embodiment 83. The therapeutic agent according to Embodiment 61, the (GIC) liquid consisting of water and an NGIC powder.

Embodiment 84. The therapeutic agent according to Embodiment 81, the NGIC powder comprising:
SiO2 0.41-2.055 wt %;
CaO 0.239-1.195 wt %;
Na₂O 0.24-1.2 wt %;
P₂O₅ 0.59-0.295 wt %;
Ag nanoparticles 0.51-0.255 wt %;
SiO₂ 33.4-41.48 wt %;
Al₂O₃19.09-28.314 wt %;
AlF₃ 1.52-2.376 wt %;
CaF₂ 14.915-19.899 wt %;
NaF 3.42-9.207 wt %; and
AlPO₄ 3.61-11.88 wt %.

Embodiment 85. The therapeutic agent according to Embodiment 81, the NGIC powder comprising:
Ag nanoparticles 0.51-0.255 wt %;
SiO₂ 33.4-41.48 wt %;
Al₂O₃19.09-28.314 wt %;
AlF₃ 1.52-2.376 wt %;
CaF₂ 14.915-19.899 wt %;
NaF 3.42-9.207 wt %; and
AlPO₄ 3.61-11.88 wt %.

Embodiment 86. The therapeutic agent according to Embodiment 81, the NGIC powder comprising:
Ag nanoparticles 0.51-0.255 wt %;
SiO₂ 33.4-41.48 wt %;
Al₂O₃19.09-28.314 wt %; and
CaF₂ 14.915-19.899 wt %.

Embodiment 87. The therapeutic agent according to Embodiment 81, the NGIC liquid comprising:
Water 45 wt %;
Polyacrylic acid 36 wt %;
Polybasic carboxylic acid 9 wt %; and
Poly (vinylphosphonic acid) 10 wt %.

Embodiment 88. The therapeutic agent according to Embodiment 86, the NGIC liquid comprising:
Water 45 wt %;
Polyacrylic acid 36 wt %;
Polybasic carboxylic acid 9 wt %; and
Poly (vinylphosphonic acid) 10 wt %.

### Materials and Methods

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1—Preparation of Test Samples for FIGS. 1 and 2

Materials: NGIC samples were prepared by hand mix for biocompatibility and mechanical strength analysis.

Methods: The NGIC material were prepared by incorporating different proportions of the poly(vinylphosphonic acid) (PVPA) (containing 1%, 5%, 10%, 20% of PVPA by weight) into the polyacrylic acid solution. The silicate glass powder was mixed with the polyacrylic acid solution at a powder/liquid ratio of 3.6/1.0 for 60 s at 23° C. and humidity of greater than 30% and less than 70%. Conventional GIC with no PVPA was used as a control. 6 samples were prepared per subgroup.

Data: 144 circular specimens of 5 mm in diameter and 2 mm in height were prepared to assess cell cytotoxicity (ISO 7405:2018). 60 specimens of 4 mm in diameter and 6 mm in height were prepared to assess mechanical strength according to ISO standards (ISO 9917-1:2007).

Analysis: For a common standard deviation of 10 with a power of 0.80 and α=0.05, the sample size was six in each subgroup.

Results: 204 specimens of NGIC material were produced for each test group according to Table 2.

TABLE 2
Composition of the provided materials of FIGS. 1-2

| | Composition/wt % | |
|---|---|---|
| | Silicate | Polyacrylic |
| Group | glass-powder | acid/PVPA |
| 0% PVPA | 100 | 100/0 |
| 1% PVPA | 100 | 99/1 |
| 5% PVPA | 100 | 95/5 |
| 10% PVPA | 100 | 90/10 |
| 20% PVPA | 100 | 80/20 |

### Example 2—Biocompatibility

Materials: 144 circular samples from Example 1 were plated in 96-wells plate for Cell Counting Kit-8 test.

Methods: The circular specimens of 5 mm in diameter and 2 mm in height were prepared to assess cell cytotoxicity (ISO 7405:2018). The specimens were sterilized using an ethylene oxide sterilizer. Human gingival fibroblast (HGF) cells were cultivated on the surface of the specimens in Dulbecco's Modified Eagle's Medium (DMEM) supplemented with 10% fetal bovine serum, with 60 μg/mL penicillin and with 100 μg/mL streptomycin in a 37° C. incubator with 5% carbon dioxide in the humidified air. The cytotoxicity of the AbGICs was determined by assessing the cells qualitatively and quantitatively (ISO 10993-5:2009). The proliferative potential of HGFs were determined by the Cell Counting Kit-8 (CCK-8) (Apexbio, MA, USA) assay. For CCK-8 assay, 3×10³ HGF cells were inoculated on the surface of the specimens in a 96-well plate. After 1, 3, 5, and 7 days of coculture, HGF cells were treated with CCK-8 regents at 37° C. for 2 h and the optical density (OD) at 450 nm was measured by a microplate reader spectramax M2 (Molecular Devices, USA).

Data: For CCK-8 assay, it was demonstrated that the optical density (OD) at 450 nm of HGF cells in NGIC group were decreased compared with the blank control group without NGIC sample (P<0.01). However, there are no statistical differences among the group addition of PVPA in NGIC and the commercial group (P>0.05).

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIG. 1, the addition of PVPA in NGIC showed similar biocompatibility as related art commercial GIC products in both biocompatibility and toxicity test.

### Example 3—Mechanical Strength

Materials: 60 samples from Example 1 were mounted in Instron testing machine to determine the compressive strength and diametral tensile strength.

Methods: The cylindrical specimens were prepared by inserting the freshly mixed cement paste into cylindrical polythene split molds of 4 mm in diameter and 6 mm in height according to ISO standards (ISO 9917-1:2007). With a static load of under a glass slide for 60 min, the specimens were removed from the molds and covered with a thin layer of petroleum jelly. After 24 h, remove the petroleum jelly on the specimens and determine the compressive strength and diametral tensile strength (DTS) with a mechanical testing device (ELECTROPULS E3000 Universal Testing System, INSTRON, MA, USA). The compressive strength (MPa) is calculated by the equation:

$$CS = \frac{4 L}{2 {D}^{2}}$$

where L is the fracture load, D is the diameter of the sample.

The DTS (MPa) is calculated by the formula:

$$DTS = \frac{2 L}{π DH}$$

where L is the fracture load (N), D is the diameter of the sample and H is the height of the sample.

Data: For the compressive strength, NGIC containing 10% PVPA showed increased compressive strength than the control group (P<0.05). However, the NGIC containing 20% PVPA showed decreased compressive strength than the control group (P<0.05). NGIC containing 1% and 5% PVPA have no differences in compressive strength compared with control group (P>0.05). For the diametral tensile strength, NGIC containing 10% PVPA showed increased diametral tensile strength than the control group (P<0.05). However, the NGIC containing 20% PVPA showed decreased diametral tensile strength than the control group (P<0.05). NGIC containing 1% and 5% PVPA have no differences in diametral tensile strength compared with control group (P>0.05).

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIG. 2, the addition of 10% PVPA enhanced the mechanical strength of NGIC in both compressive strength test and diametral tensile strength test.

### Example 4—Preparation of Test Samples for FIG. 3

Materials: The Nanosilver bioactive glass is synthesized by the sol-gel method for SEM and EDX analysis.

Methods: 1. 5 g of 45S5 bioactive glass was prepared. A 7.31 g of TEOS was added to 1 M nitric acid with a molar ration of H₂O:TEOS=18:1. The nitric acid was prepared by mixing 1.7 ml 69% nitric and 25 ml DI water. The mixture was stirred for 2 h until a clear sol was formed. A 0.77 g TEP was added. A 5.17 g Ca (NO3)₂ was then added. A 3.375 g NaNO₃ was added. Finally, a 0.415 g AgNO₃ was added. The mixture was stirred until all chemicals were dissolved. The sol mixture was placed in a microwave oven under microwave irradiation for 15 min with the power of 800 W. The mixture was dried overnight at 80° C. The dried gel was calcined at 700° C. for 3 h. The solid collected was ground to obtain a fine powder.

45S5 bioactive glass=Ag:SiO₂:Na₂O:CaO:P₂O₅=3:43.1:24.4:26.9:2.6 (mole ratio)

TABLE 3
45S5 bioactive glass composition.

| Molar ratio of | TEOS | TEP | Ca(NO₃)₂•4H₂O | NaNO₃ | AgNO₃ | HNO₃ | Water |
|---|---|---|---|---|---|---|---|
| Ag:Si:Na:Ca:P | (g) | (g) | (g) | (g) | (g) | (ml) | (ml) |
| Ag:Si:Na:Ca:P | 7.31 | 0.77 | 5.17 | 3.375 | 0.415 | 0.9 | 12.5 |
| 3:43.1:24.4:26.9:2.6 | | | | | | | |

Results: 5 grams 45S5 bioactive glass powder were set aside for SEM and EDX analysis.

### Example 5—SEM and EDX

Materials: 0.1 gram samples from Example 4 were observed under scanning electron microscopy (SEM) and energy-dispersive X-ray spectroscopy (EDX) under SEM.

Methods: The surface morphologies of the 45S5 bioactive glass powder were observed under scanning electron microscopy (SEM) (Hitachi 5-4800 FEG Scanning Electron Microscope, Hitachi Ltd., Tokyo, Japan) at 5 kV in high-vacuum mode. An elemental assessment was carried out on the surfaces of six specimens in each group. The levels of ions were evaluated by energy-dispersive X-ray spectroscopy (EDX) under SEM. The elemental assessment was performed by measuring five 5×5 μm² square areas on the enamel surface in each sample.

Data: The SEM micrographs demonstrated that the bioactive glass consists of nanoparticles with an irregular shape particle. The EDX confirms the presence of Ag, Si, Na, Ca and P in the glass sample as prepared.

Results: As shown in FIG. 3, embodiments of the subject invention provide a novel nanosilver bioactive glass (NanoAg BAG).

### Example 6—Preparation of Test Samples for FIGS. 4 Through 9

Materials: NGIC samples were prepared were prepared by hand mix for biocompatibility and mechanical strength analysis, fluoride ion release, calcium, phosphate and silver release analysis.

Methods: The NGIC material were prepared by incorporating different proportions of the NanoAg BAG (containing 0%, 1%, 2%, 5% of NanoAg BAG by weight) the silicate glass powder. The silicate glass powder was mixed with the polyacrylic acid containing 10% poly(vinylphosphonic acid) (PVPA) at a powder/liquid ratio of 3.6/1.0 for 60 s at 23° C. and humidity of greater than 30% and less than 70%. Conventional GIC with no PVPA and NanoAg BAG was used as a control. 6 samples were prepared per subgroup.

Data: 144 circular specimens of 5 mm in diameter and 2 mm in height were prepared to assess cell cytotoxicity (ISO 7405:2018). 60 specimens of 4 mm in diameter and 6 mm in height were prepared to assess mechanical strength according to ISO standards (ISO 9917-1:2007). 30 circular specimens of 5 mm in diameter and 2 mm in height were prepared to assess the fluoride ion release. 30 circular specimens of 5 mm in diameter and 2 mm in height were prepared to assess the calcium, phosphate, and silver release.

Analysis: For a common standard deviation of 10 with a power of 0.80 and α=0.05, the sample size was six in each group.

Results: 264 circular samples of NGIC material were produced for each test group according to Table 4.

TABLE 4
Composition of the provided materials of FIGS. 4-11

| | Composition/wt % | |
|---|---|---|
| | Silicate glass-powder/ | Polyacrylic |
| Group | NanoAg BAG | acid/PVPA |
| 0% NanoAg BAG + 0% PVPA | 100/0 | 100/0 |
| 0% NanoAg BAG + 10% PVPA | 100/0 | 90/10 |
| 1% NanoAg BAG + 10% PVPA | 99/1 | 90/10 |
| 2% NanoAg BAG + 10% PVPA | 98/2 | 90/10 |
| 5% NanoAg BAG + 10% PVPA | 95/5 | 90/10 |

### Example 7—Biocompatibility

Materials: 144 samples from Example 6 were plated in 96-wells plate for Cell Counting Kit-8 test.

Methods: The circular specimens of 5 mm in diameter and 2 mm in height were prepared to assess cell cytotoxicity (ISO 7405:2018). The specimens were sterilized using an ethylene oxide sterilizer. Human gingival fibroblast (HGF) cells were cultivated on the surface of the specimens in Dulbecco's Modified Eagle's Medium (DMEM) supplemented with 10% fetal bovine serum, with 60 μg/mL penicillin and with 100 μg/mL streptomycin in a 37° C. incubator with 5% carbon dioxide in the humidified air. The cytotoxicity of the NGICs was determined by assessing the cells qualitatively and quantitatively (ISO 10993-5:2009). The proliferative potential of HGFs were determined by the Cell Counting Kit-8 (CCK-8) (Apexbio, MA, USA) assay. For CCK-8 assay, 3×10³ HGF cells were inoculated on the surface of the specimens in a 96-well plate. After 1, 3, 5, and 7 days of coculture, HGF cells were treated with CCK-8 regents at 37° C. for 2 h and the optical density (OD) at 450 nm was measured by a microplate reader spectramax M2 (Molecular Devices, USA).

Data: For CCK-8 assay, it was demonstrated that the optical density (OD) at 450 nm of HGF cells in NGIC group were decreased compared with the blank control group without NGIC sample (P<0.01). However, there are no statistical differences among the group addition of NanoAg BAG and PVPA in NGIC and the commercial group (0% NanoAg BAG+0% PVPA) (P>0.05).

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIG. 4, the addition of NanoAg BAG and PVPA showed similar biocompatibility as related art GIC product (0% NanoAg BAG+0% PVPA) for 24 h. 1% NanoAg BAG and 10% PVPA has similar biocompatibility as related art GIC product.

### Example 8—Mechanical Strength

Materials: 60 samples from Example 6 were mounted in Instron testing machine to determine the compressive strength and diametral tensile strength.

Methods: The cylindrical specimens were prepared by inserting the freshly mixed cement paste into cylindrical polythene split molds of 4 mm in diameter and 6 mm in height according to ISO standards (ISO 9917-1:2007). With a static load of under a glass slide for 60 min, the specimens were removed from the molds and covered with a thin layer of petroleum jelly. After 24 h, remove the petroleum jelly on the specimens and determine the compressive strength and diametral tensile strength (DTS) with a mechanical testing device (ELECTROPULS E3000 Universal Testing System, INSTRON, MA, USA). The compressive strength (MPa) is calculated by the equation:

$$CS = \frac{4 L}{2 {D}^{2}}$$

where L is the fracture load, D is the diameter of the sample.

The DTS (MPa) is calculated by the formula:

$$DTS = \frac{2 L}{π DH}$$

where L is the fracture load (N), D is the diameter of the sample and H is the height of the sample.

Data: For the compressive strength, the addition of 1% NanoAg BAG+10% PVPA showed the highest compressive strength among all the group and showed increased compressive strength than the commercial group (0% NanoAg BAG+0% PVPA) (P<0.01). The addition of 0% NanoAg BAG+10% PVPA and 2% NanoAg BAG+10% PVPA showed increased compressive strength than the commercial group (0% NanoAg BAG+0% PVPA) (P<0.01). The addition of 5% NanoAg BAG+10% PVPA showed no statistical differences in compressive strength than the commercial group (0% NanoAg BAG+0% PVPA) (P>0.05).

For the diametral tensile strength, the addition of 1% NanoAg BAG+10% PVPA showed the highest diametral tensile strength among all the group and showed increased diametral tensile strength than the commercial group (0% NanoAg BAG+0% PVPA) (P<0.01). The addition of 0% NanoAg BAG+10% PVPA and 2% NanoAg BAG+10% PVPA showed increased diametral tensile strength than the commercial group (0% NanoAg BAG+0% PVPA) (P<0.01). The addition of 5% NanoAg BAG+10% PVPA showed no statistical differences in diametral tensile strength than the commercial group (0% NanoAg BAG+0% PVPA) (P>0.05).

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIG. 5, the addition of 1% NanoAg BAG+10% PVPA enhanced the mechanical strength of NGIC in both a compressive strength test and a diametral tensile strength test.

### Example 9—Fluoride Release

Materials: 30 circular samples from Example 6 were stored in 24-well plates for fluoride release test.

Methods: 30 disc-shaped specimens of 5 mm in diameter and 2 mm in thickness. Fluoride ion release was recorded for 14 days. Each specimen was immersed in a capped polystyrene bottle containing 2 mL of distilled water (pH 7.0) individually. The specimens were stored at 37° C. for a total immersion time of 14 days. Fluoride concentration in storage solution were measured at different intervals using a fluoride ion-selective electrode (Oakton, Cole Parmer, IL, USA) connected to an ion analyzer (Oakton 510 ion series, Cole Parmer, IL, USA). The electrode was calibrated to external standards of 0.1, 1, 10, 100, 1000 μg/g F. A volume of 2 mL of a total ionic strength adjustment buffer TISAB II (Thermo Fisher Scientific, Chelmsford, USA) was added to the storage solution before fluoride ion measurements to increase the ionic strength in the storage solution, and hence, the accuracy of readings. With any potential drifts in the electrode measurements, the electrode was re-calibrated using the standards. The coefficient of determination for all calibrations were >0.99. A standard curve was plotted against the standards with the auto-determined mV potential, which was further used to derive F concentration in test solutions.

Data: The fluoride release levels of the NGIC materials were higher on the first day. Subsequently, the daily fluoride release of the NGIC materials gradually decreased until day 7, and the stable low-level fluoride release state was maintained from 7 to 14 days. Among them, the addition of 5% NanoAg BAG+10% PVPA had the highest fluoride release, which was higher than commercial group (0% NanoAg BAG+0% PVPA), and the difference was statistically significant (P<0.05). Subsequently, the addition of 1% NanoAg BAG+10% PVPA and 2% NanoAg BAG+10% PVPA NGIC group had higher fluoride release than commercial group (0% NanoAg BAG+0% PVPA), and the difference was statistically significant (P<0.05). The addition of 0% NanoAg BAG+10% PVPA NGIC group showed the same fluoride release with the commercial group (0% NanoAg BAG+0% PVPA) (P>0.05).

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIG. 6, compared to related art GIC, NGIC had higher fluoride release, which can promote tooth remineralization and prevent, reduce, or inhibit dental caries.

### Example 10—Phosphate, Calcium and Silver Release

Materials: 30 samples from Example 6 were stored in 15 mL tubes for calcium, phosphate and silver release test.

Methods: The Ag detection was performed using Inductively coupled plasma-optical emission spectrometry (ICP-OES) (Spectro arcos, Germany). Prepare a series of standard solutions from the 1000 μg/mL national standard solutions of calcium, phosphate, and silver (GSB 04-1712-2004, China), and then make the standard curve of each metal element by measuring them separately. Prepare of silver standard solution: Pipette different volumes of silver standard stock solution with a concentration of 1000 μg/mL into the volumetric flask, the standard solution is diluted into a series of different calcium, phosphate, and silver ion solution, the concentration is 0, 10015625, 0.03125, 0.0625, 0.125, 0.25, 0.5, 1, 5 mg/L calcium, phosphate, and silver. Use 4 mL of test solution for one sample. First, put the sample introduced in the ICP-OES used in the experiment into distilled water to flush the pipeline; then, draw the prepared calcium, phosphate, and silver ion standard solution separately, and use the obtained data to draw the working curve of calcium, phosphate, and silver ion. According to the experimental measurement results of the calcium, phosphate, and silver element standard solution, a standard curve diagram is drawn from the ion content corresponding to the emission intensity, and then linear fitting is performed to finally obtain the calcium, phosphate, and silver element standard curve regression equation and correlation coefficient. Each test solutions were estimated for calcium, phosphate, and silver concentrations in triplicate.

Data: For phosphate release, first 7 days (Day 7) showed higher phosphate release than second 7 days (Day 14). During day 7 and 14, the addition of 5% NanoAg BAG+10% PVPA had the highest phosphate release, which was higher than commercial group (0% NanoAg BAG+0% PVPA), and the difference was statistically significant (P<0.01). The addition of addition of 0% NanoAg BAG+10% PVPA, 10% NanoAg BAG+10% PVPA and 2% NanoAg BAG+10% PVPA NGIC group had no statistical differences in phosphate release compared with the commercial group (0% NanoAg BAG+0% PVPA) (P>0.05).

For calcium release, first 7 days (Day 7) showed higher calcium release than second 7 days (Day 14). During day 7 and 14, the addition of 5% NanoAg BAG+10% PVPA had the highest calcium release, which was higher than commercial group (0% NanoAg BAG+0% PVPA), and the difference was statistically significant (P<0.01). The addition of 0% NanoAg BAG+10% PVPA, 1% NanoAg BAG+10% PVPA, 2% NanoAg BAG+10% PVPA NGIC group and the commercial group (0% NanoAg BAG+0% PVPA) NGIC cannot detect the calcium release.

For silver release, first 7 days (Day 7) showed higher calcium release than second 7 days (Day 14). During day 7 and 14, the addition of 5% NanoAg BAG+10% PVPA had the highest calcium release, which was higher than commercial group (0% NanoAg BAG+0% PVPA), and the difference was statistically significant (P<0.01). The addition of 1% NanoAg BAG+10% PVPA and 2% NanoAg BAG+10% PVPA NGIC showed higher silver release than commercial group (0% NanoAg BAG+0% PVPA), and the difference was statistically significant (P<0.01). The addition of addition of 0% NanoAg BAG+10% PVPA and 1% NanoAg BAG+10% PVPA NGIC cannot detect the silver release.

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIGS. 7-8, NGICs released calcium and phosphate, which can promote tooth remineralization and prevent, reduce, or inhibit dental caries. As shown in FIG. 9, NGIC released a higher concentration of silver at Day 7, which can inhibit the growth of oral bacteria.

### Example 11—Compositions

An exemplary composition for one embodiment of the subject invention is provided in Table 5 and Table 6. The ratio of nanosilver bioactive glass (NBG) to NGIC in Table 6 includes compositions ranging from 1% NBG and 99% NGIC, to 5% NBG and 95% NGIC. The NGIC Powder components were mixed separately from the NGIC Liquid components, before combining the two to form the NGIC. The NGIC and NBG were then combined to form a therapeutic composition for dental use.

TABLE 5
The composition of Nanosilver bioactive glass (NBG)

| Materials | Composition |
|---|---|
| SiO₂ | 41.1% |
| CaO | 23.9% |
| Na₂O | 24.0% |
| P₂O₅ | 5.9% |
| Ag nanoparticles | 5.1% |

TABLE 6
The composition of NGIC
Ratio of (NBG:NGIC) can be
from (1%:99%) to (5%:95%) by weight

| NGIC | Materials | Weight percentages | |
|---|---|---|---|
| NGIC Powder | SiO2 | 0.32-1.61 | wt % |
| | CaO | 0.19-0.94 | wt % |
| | Na₂O | 0.19-0.94 | wt % |
| | P₂O₅ | 0.46-0.23 | wt % |
| | Ag nanoparticles | 0.39-0.20 | wt % |
| | SiO₂ | 24.83-30.83 | wt % |
| | Al₂O₃ | 14.19-21.05 | wt % |
| | AlF₃ | 1.13-1.77 | wt % |
| | CaF₂ | 11.09-17.96 | wt % |
| | NaF | 2.54-6.85 | wt % |
| | AlPO₄ | 2.68-8.84 | wt % |
| NGIC liquid | H₂O | 9.78 | wt % |
| with 10% | Polyacrylic acid | 7.82 | wt % |
| PVPA | Polybasic carboxylic acid | 1.95 | wt % |
| | Poly (vinylphosphonic acid) | 2.17 | wt % |

TABLE 7
The composition of various components of NGIC

| NGIC | Materials | Weight percentages | |
|---|---|---|---|
| Nanosilver | SiO2 | 0.41-2.055 | wt % |
| bioactive glass | CaO | 0.239-1.195 | wt % |
| | Na₂O | 0.24-1.2 | wt % |
| | P₂O5 | 0.59-0.295 | wt % |
| | Ag nanoparticles | 0.51-0.255 | wt % |
| Silicate glass- | SiO₂ | 33.4-41.48 | wt % |
| powder | Al₂O₃ | 19.09-28.314 | wt % |
| | AlF₃ | 1.52-2.376 | wt % |
| | CaF₂ | 14.915-19.899 | wt % |
| | NaF | 3.42-9.207 | wt % |
| | AlPO₄ | 3.61-11.88 | wt % |
| NGIC liquid with | H₂O | 45 | wt % |
| 10% PVPA | Polyacrylic acid | 36 | wt % |
| | Polybasic carboxylic | 9 | wt % |
| | acid | | |
| | Poly (vinylphosphonic | 10 | wt % |
| | acid) | | |

### Example 12—Properties

The compressive strengths of the provided NGIC measured for embodiments of the subject invention are higher than the compressive strength values reported in related art. In addition to improved strength, complimentary benefits of certain embodiments of the subject invention include but are not limited to the following.

The GIC taught in related art has antibacterial effect against a limited number (e.g., four different strains) of bacteria. In embodiments of the subject invention, the release of silver ions from NGIC was evaluated in deionized water for 7 d and 14 d, showing broad spectrum antibacterial effect beyond and in contrast to that reported for related art.

The bioactive glass GIC taught in related art is claimed to have antibacterial effect. This is in some cases due to a pH change after immersion in physiologic saline. In embodiments of the subject invention, the antibacterial effect of NGIC is due to the nanosilver, advantageously providing an alternative mode of action for improved antibacterial effect.

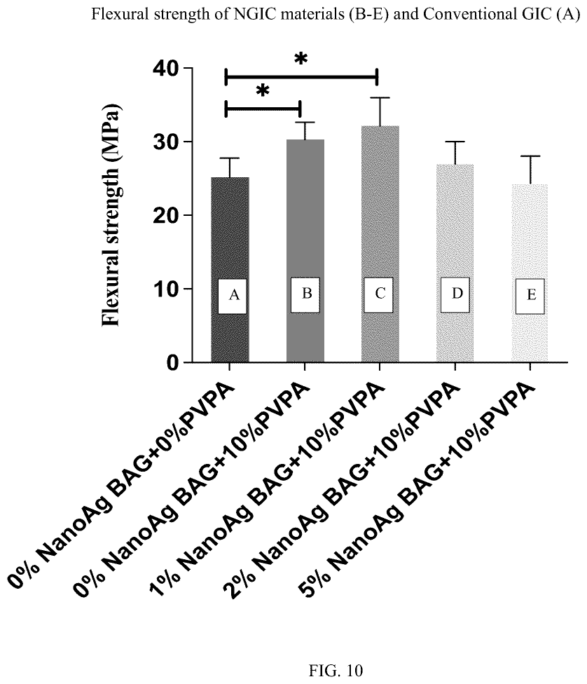

### Example 13—Preparation of Test Samples for FIG. 10

Materials: NGIC samples were prepared by hand mix for flexural strength analysis.

Methods: The NGIC material were prepared by incorporating different proportions of the NanoAg BAG (containing 0%, 1%, 2%, 5% of NanoAg BAG by weight) the silicate glass powder. The silicate glass powder was mixed with the polyacrylic acid containing 10% poly(vinylphosphonic acid) (PVPA) at a powder/liquid ratio of 3.6/1.0 for 60 s at 23° C. and humidity of greater than 30% and less than 70%. Conventional GIC with no PVPA and NanoAg BAG was used as a control. 6 samples were prepared per subgroup.

Data: 30 rectangular samples of 25 mm in length, 2 mm in thickness and width were prepared to assess the flexural strength according to the ISO standards (ISO 4049:2019).

Analysis: For a common standard deviation of 10 with a power of 0.80 and α=0.05, the sample size was six in each subgroup.

Results: 30 rectangular samples of NGIC material were produced for each test group according to Table 4.

### Example 14—Flexural Strength

Materials: 30 samples from Example 13 were mounted in Instron testing machine to determine the flexural strength.

Methods: Split stainless-steel moulds (25 mm in length and 2 mm in width and height) were used to prepare the rectangular samples according to the ISO standards (ISO 4049:2019). With a static load of under a glass slide for 60 min, the samples were removed from the molds and covered with a thin layer of petroleum jelly. After 24 h, remove the petroleum jelly on the samples and determine the flexural strength (FS) with a mechanical testing device (ELECTROPULS E3000 Universal Testing System, INSTRON, Norwood, MA, USA). The flexural strength (MPa) is calculated by the equation:

$$FS = \frac{3 FL}{2 {bd}^{2}}$$

where F is the load (force) at the fracture point (MPa), L is the length of the support span, b is width, d is thickness of the samples.

Data: For the flexural strength, the addition of 1% NanoAg BAG+10% PVPA showed the highest flexural strength among all the group and showed increased flexural strength than the commercial group (0% NanoAg BAG+0% PVPA) (P<0.05). The addition of 0% NanoAg BAG+10% PVPA showed increased flexural strength than the commercial group (0% NanoAg BAG+0% PVPA) (P<0.05). The addition of 2% NanoAg BAG+10% PVPA and 5% NanoAg BAG+10% PVPA showed no statistical differences in flexural strength than the commercial group (0% NanoAg BAG+0% PVPA) (P>0.05).

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIG. 10, the addition of 0% NanoAg BAG+10% PVPA and 1% NanoAg BAG+10% PVPA enhanced the flexural strength of NGIC.

FIG. 10 shows the results of the flexural strength in various groups of materials according to certain embodiments of the subject invention. (*p<0.01).

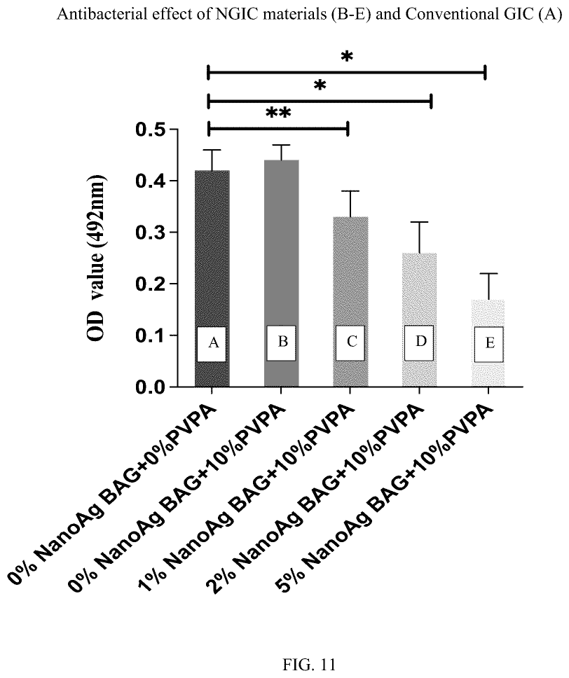

### Example 15—Preparation of Test Samples for FIGS. 11 and 12

Materials: NGIC samples were prepared were prepared for antibacterial effect test by biofilm metabolic activity test.

Methods: The NGIC material were prepared by incorporating different proportions of the NanoAg BAG (containing 0%, 1%, 2%, 5% of NanoAg BAG by weight) the silicate glass powder. The silicate glass powder was mixed with the polyacrylic acid containing 10% poly(vinylphosphonic acid) (PVPA) at a powder/liquid ratio of 3.6/1.0 for 60 s at 23° C. and humidity of greater than 30% and less than 70%. Conventional GIC with no PVPA and NanoAg BAG was used as a control. 6 samples were prepared per subgroup.

Data: 30 circular samples of 5 mm in diameter and 2 mm in height were prepared to assess antibacterial test.

Analysis: For a common standard deviation of 10 with a power of 0.80 and α=0.05, the sample size was six in each group.

Results: 30 circular samples of NGIC material were produced for each test group according to Table 4.

### Example 16—Absorbance of Optical Density (OD) at 492 nm and Antibacterial Test-Biofilm Metabolic Activity

Materials: 30 samples from Example 15 were soaked in S. mutans culture for biofilm metabolic activity test.

Methods: Teflon moulds (5 mm in diameter and 2 mm in height) were used to prepare the circular samples to assess the antibacterial effect. The Streptococcus mutans (S. mutans) American Type Culture Collection (ATCC) UA159 was used in this study. Each specimen was soaked in a 1-mL S. mutans culture of 10⁷ cells/mL in a brain-heart-infusion broth. The samples were anaerobically incubated at 37° C. for 2 days. The metabolic activity of S. mutans biofilm on the samples were evaluated by the XTT (2,3-(2-methoxy-4-nitro-5-sulphophenyl)′5-[(phenylamino) carbonyl] [symbol] 2H-tetrazolium hydroxide) (Sigma, Aldrich) reduction assay. After 2 days of incubation, 100 μL of XTT reagent solution (AppliChem GmbH, Darmstadt, Germany) was added to each well, and the plate was incubated at 37° C. for 3 h. Then collected solution and centrifugated for 10 min at 13,200 rpm to remove the debris. The absorbance of optical density (OD) was then measured at 492 nm using a microplate reader.

Data: Biofilm metabolic activity assay showed that the absorbance of 0% NanoAg BAG+0% PVPA, 0% NanoAg BAG+10% PVPA, 1% NanoAg BAG+10% PVPA, 2% NanoAg BAG+10% PVPA and 5% NanoAg BAG+10% PVPA were 0.42±0.04, 0.44±0.03, 0.33±0.05, 0.26+0.05 and 0.17±0.05, respectively. Biofilm in 1% NanoAg BAG+10% PVPA, 2% NanoAg BAG+10% PVPA and 5% NanoAg BAG+10% PVPA surfaces showed decreased biofilm metabolic activity than the commercial group (0% NanoAg BAG+0% PVPA) (P<0.05). The addition of 0% NanoAg BAG+10% PVPA showed no statistical differences in biofilm than the commercial group (0% NanoAg BAG+0% PVPA) (P>0.05).

Analysis: The quality data were analyzed using SPSS Statistics 20 (IBM Corporation, Somers, NY, USA). Quantitative data were expressed as mean±standard deviation. Between-group differences were determined using one-way ANOVA. The cutoff level was set at 5% significance.

Results: As shown in FIG. 11, the addition of 1% NanoAg BAG+10% PVPA, 2% NanoAg BAG+10% PVPA and 5% NanoAg BAG+10% PVPA reduced bacteria metabolic activity, showing enhanced the antibacterial effect of NGIC.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

All patents, patent applications, provisional applications, and publications referred to or cited herein are incorporated by reference in their entirety, including all figures and tables, to the extent they are not inconsistent with the explicit teachings of this specification.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

When ranges are used herein, such as for dose ranges, combinations and subcombinations of ranges (e.g., subranges within the disclosed range), specific embodiments therein are intended to be explicitly included.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Where particular values are described in the application and claims, unless otherwise stated the term “about” meaning within an acceptable error range for the particular value should be assumed. In the context of compositions containing amounts of concentrations of ingredients where the term “about” is used, these values include a variation (error range) of 0-10% around the value (X±10%).

As used herein, each of n, N, X and Y is intended to include ≥1, ≥2, ≥3, ≥4, ≥5, ≥6, ≥7, ≥8, ≥9, ≥10, ≥11, ≥12, ≥13, ≥14, ≥15, ≥16, ≥17, ≥18, ≥19, ≥20, ≥21, ≥≥22, ≥≥23, ≥≥24, ≥≥25, ≥≥26, ≥≥27, ≥≥28, ≥≥29, and ≥30.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### REFERENCES

1. Hoshika, S., et al., Effect of conditioning and 1 year aging on the bond strength and interfacial morphology of glass-ionomer cement bonded to dentin. Dent Mater, 2021. 37(1): p. 106-112.
2. Agha, A., S. Parker, and M. P. Patel, Development of experimental resin modified glass-ionomer cements (RMGICs) with reduced water uptake and dimensional change. Dent Mater, 2016. 32(6): p. 713-22.
3. Friedl, K., K. A. Hiller, and K. H. Friedl, Clinical performance of a new glass-ionomer based restoration system: a retrospective cohort study. Dent Mater, 2011. 27(10): p. 1031-7.
4. Gemeinhart, R. A., et al., Osteoblast-like cell attachment to and calcification of novel phosphonate-containing polymeric substrates. Journal of Biomedical Materials Research Part A: An Official Journal of The Society for Biomaterials, The Japanese Society for Biomaterials, and The Australian Society for Biomaterials and the Korean Society for Biomaterials, 2006. 78(3): p. 433-440.
5. Heck, K., et al., Six-year results of a randomized controlled clinical trial of two glass-ionomer cements in class II cavities. J Dent, 2020. 97: p. 103333.
6. Espinosa-Cristóbal, L. F., et al., Antimicrobial and Substantivity Properties of Silver Nanoparticles against Oral Microbiomes Clinically Isolated from Young and Young-Adult Patients. Journal of Nanomaterials, 2019. 2019: p. 3205971.
7. Nizami, M. Z. I., et al., Metal and Metal Oxide Nanoparticles in Caries Prevention: A Review. Nanomaterials (Basel), 2021. 11(12).
8. Hench, L. L., The story of Bioglass®. Journal of Materials Science: Materials in Medicine, 2006. 17(11): p. 967-978.
9. Banerjee, A., et al., An in vitro evaluation of selective demineralised enamel removal using bio-active glass air abrasion. Clinical Oral Investigations, 2011. 15(6): p. 895-900.
10. Yang, S. Y., et al., Acid neutralizing, mechanical and physical properties of pit and fissure sealants containing melt-derived 45S5 bioactive glass. Dent Mater, 2013. 29(12): p. 1228-35.

We claim:

1. A novel glass-ionomer cement (NGIC), comprising:
a silicate glass powder;
a polyacrylic acid powder;
a poly(vinylphosphonic acid) (PVPA) powder;
a nanosilver bioactive glass; and
a polyacrylic acid solution.

2. The NGIC according to claim 1, wherein the silicate glass-powder comprises calcium-alumino-fluorosilicate glass.

3. The NGIC according to claim 2, wherein the silicate glass-powder is calcium-alumino-fluorosilicate glass.

4. The NGIC according to claim 1, wherein the wt % ratio of (polyacrylic acid)/PVPA is greater than or equal to 90/10.

5. The NGIC according to claim 4, wherein the wt % ratio of (polyacrylic acid)/PVPA is greater than 95/5.

6. The NGIC according to claim 5, wherein the wt % ratio of (polyacrylic acid)/PVPA is greater than 99/1.

7. The NGIC according to claim 1, wherein the wt % ratio of (silicate glass powder)/(nanosilver bioactive glass) is greater than or equal to 95/5.

8. The NGIC according to claim 7, wherein the wt % ratio of (silicate glass powder)/(nanosilver bioactive glass) is greater than 98/2.

9. The NGIC according to claim 8, wherein the wt % ratio of (silicate glass powder)/(nanosilver bioactive glass) is greater than 99/1.

10. The NGIC according to claim 1, wherein the wt % ratio of (polyacrylic acid)/PVPA is about 90/10, and the wt % ratio of (silicate glass powder)/(nanosilver bioactive glass) is greater than 95/5, but less than or equal to 99/1.

11. A method of preparing a NGIC, comprising combining a liquid mixture of polyacrylic acid and PVPA with a powdered mixture of silicate glass powder, polyacrylic acid powder, and nanosilver bioactive glass.

12. The method according to claim 11, wherein the silicate glass powder comprises calcium-alumino-fluorosilicate glass.

13. The method according to claim 12, wherein the silicate glass powder is calcium-alumino-fluorosilicate glass.

14. The method according to claim 11, wherein the wt % ratio of (polyacrylic acid)/PVPA is greater than 90/10.

15. The method according to claim 11, wherein the wt % ratio of (silicate glass powder)/(nanosilver bioactive glass) is greater than or equal to 95/5.

16. The method according to claim 11, wherein the wt % ratio of (polyacrylic acid)/PVPA is about 90/10, and the wt % ratio of (silicate glass powder)/(nanosilver bioactive glass) is greater than or equal to 95/5, but less than or equal to 99/1.

17. A novel glass-ionomer cement (NGIC), comprising:
a silicate glass powder;
a polyacrylic acid powder;
a poly(vinylphosphonic acid) (PVPA) powder;
a nanosilver bioactive glass; and
a polyacrylic acid solution;
wherein the silicate glass-powder is calcium-alumino-fluorosilicate glass;
wherein the wt % ratio of (polyacrylic acid)/PVPA is greater than or equal to 90/10; and
wherein the wt % ratio of (silicate glass powder)/(nanosilver bioactive glass) is greater than or equal to 95/5.

18. The NGIC according to claim 17, the nanosilver bioactive glass comprising a population of nanosilver particles, a majority of which are between 8-15 nm in size.

19. The NGIC according to claim 17, the nanosilver bioactive glass comprising a population of bioactive glass particles, a majority of which are between 5-120 nm in size.

20. The NGIC according to claim 18, the nanosilver bioactive glass comprising a population of bioactive glass particles, a majority of which are between 5-120 nm in size.
